(19) United States
(12) Patent Application Publication
(10) Pub. No.: US 2013/0172303 A1
(43) Pub. Date: Jul. 4, 2013

(54) ROFLUMILAST COMPOSITIONS FOR THE TREATMENT OF COPD

(71) Applicant: Forest Laboratories Holdings Ltd., Hamilton (BM)

(72) Inventors: Jose Freire, Westfield, NJ (US); Xiaozhong Qian, Pennington, NJ (US); Colin Scott, Florham Park, NJ (US)

(73) Assignee: FOREST LABORATORIES HOLDINGS LTD., Hamilton (BM)

(21) Appl. No.: 13/733,187

(22) Filed: Jan. 3, 2013

Related U.S. Application Data
(60) Provisional application No. 61/582,564, filed on Jan. 3, 2012.

(51) Int. Cl. A61K 31/44; A61K 31/47; A61K 31/573

(52) U.S. Cl. CPC A61K 31/44; A61K 31/573; A61K 31/47
USPC 514/171; 514/311

(57) ABSTRACT

The present invention relates to pharmaceutical compositions comprising roflumilast in combination with a corticosteroid and/or a leukotriene receptor antagonist. The invention also relates to the use of such compositions for the treatment of Chronic Obstructive Pulmonary Disorder (COPD).

### CROSS-REFERENCE TO RELATED APPLICATIONS

This application claims priority under 35 U.S.C. §119 to U.S. Provisional Application No. 61/582,564, filed on Jan. 3, 2012, the contents of which are incorporated herein by reference.

Each of the applications and patents cited in this text, as well as each document or reference cited in each of the applications and patents (including during the prosecution of each issued patent; “application cited documents”), and each of the U.S. and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references), as well as each document or reference cited in each of the herein-cited references (including any manufacturer's specifications, instructions, etc.), is hereby expressly incorporated herein by reference. Documents incorporated by reference into this text may be employed in the practice of the invention.

### FIELD OF THE INVENTION

The present invention relates to pharmaceutical compositions comprising roflumilast in combination with a corticosteroid and/or a leukotriene receptor antagonist, and the use of such compositions for the treatment of Chronic Obstructive Pulmonary Disorder (COPD).

### BACKGROUND OF THE INVENTION

COPD is characterized by a progressive limitation of the airflow in the lungs. In North America, between three- and seven-million people are diagnosed with COPD each year, and this disease is presently the fourth leading cause of death in developed countries.

Anti-inflammatory corticosteroids have shown little effect on the loss of lung function that occurs in COPD. Neither inhaled nor oral corticosteroids suppress the inflammation in the lungs, and alveolar macrophages seem to be steroid resistant. In addition, it has been suggested that the neutrophilic inflammation which characterises COPD is insensitive to steroids, is different from the eosinophilic inflammation that characterises asthma.

Phosphodiesterase 4 (PDE4) inhibitors produce airway smooth muscle relaxation by preventing the breakdown of adenosine cyclic 3′,5′-monophosphate (cAMP).

Roflumilast is an oral PDE4 inhibitor approved by the FDA for reduction of exacerbations and improvement of lung function in COPD patients.

Another option for treating COPD is the use of combination therapies. For example, the combination of leukotriene type D₄ (LTD₄) antagonists with PDE4 inhibitors is disclosed in WO02/038155 and WO03/024488.

However, there is a need to find better options for treating COPD which would involve restoring the steroid sensitivity in subjects. Combination therapies involving the use of corticosteroids with PDE4 inhibitors for COPD are disclosed in WO01/32127, WO04/067006, WO01/19373 and WO98/41232. The activity of corticosteroid combination therapies has also been reported in the scientific literature. For example, Calverley et al., Am J Crit Care Medicine 176: 154-161, (2007); Milara et al., J Clin Experimental Allergy 41:535-548, (2011); Ford P. Chest 137:1338-1344, (2010); Newton R. Pharmacol Therapeutics 125:286-327, (2010); and Giembycz, Phosphodiesterase as Drug Targets, Handbook of Experimental Pharmacology, Springer Verlag Heidelberg 204:415-447.

### SUMMARY OF THE INVENTION

In one aspect, the present invention provides for a pharmaceutical composition comprising roflumilast in combination with a corticosteroid and, optionally or alternatively, with a leukotriene receptor antagonist. In one embodiment, the corticosteroid is selected from a group consisting of prednisone, dexamethasone and budesonide. In another embodiment, the leukotriene receptor antagonist is montelukast. In some embodiments, the pharmaceutical composition comprises 500 μg of roflumilast. In other embodiments, the pharmaceutical composition comprises 10 mg of the leukotriene receptor antagonist.

The present invention also provides an oral dosage form comprising the pharmaceutical compositions described herein.

In another aspect, the present invention provides for methods of treating COPD by administering a pharmaceutical composition or oral dosage form comprising roflumilast in combination with a corticosteroid and/or a leukotriene receptor antagonist. In some embodiments, the pharmaceutical composition or oral dosage form comprises 500 μg of roflumilast. The corticosteroid may be selected from the group consisting of dexamethasone, prednisone and budesonide. In other embodiments, the leukotriene receptor antagonist is montelukast. In some embodiments, the pharmaceutical composition or oral dosage form comprises 10 mg of montelukast.

In yet another aspect, the present invention provides a method of restoring steroid sensitivity in a subject suffering from COPD by administering to a subject an oral dosage form comprising roflumilast in combination with a corticosteroid.

These and other embodiments are disclosed or are obvious from and encompassed by the following Detailed Description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 depicts dose-inhibition curves of lipopolysaccharide (LPS) or cigarette smoke extract (CSE) induced interleukin-8 (IL-8) release in human neutrophils.

FIG. 2 depicts dose-inhibition curves of LPS or CSE induced matrix metallopeptidase-9 (MMP-9) release in human neutrophils.

FIG. 3 depicts dose-inhibition curves of LPS or CSE induced interleukin-113 (IL-113) release in human neutrophils.

FIG. 4 depicts dose-inhibition curves of LPS or CSE induced granulocyte-macrophage colony stimulating factor (GM-CSF) release in human neutrophils.

FIG. 5 depicts dose-inhibition curves of LPS or CSE induced chemokine (C—C motif) ligand 5 (CCL-5) release in human neutrophils.

FIG. 6 depicts bar graphs illustrating basal expression of the glucocorticoid resistance markers MKP-1, MIF, phosphoinositol 3-kinase 6, HDAC2, and ABCB1 in peripheral blood neutrophils from healthy subjects and patients suffering from COPD.

FIG. 7 depicts bar graphs showing expression of glucocorticoid receptors α and β in peripheral blood neutrophils from healthy subjects and patients suffering from COPD.

FIG. 8 is a graph showing the expression levels of PDE4 isoforms in neutrophils from COPD patients.

FIG. 9 illustrate graphs showing the correlation of MIF, PI3Kδ, MKP1, HDAC2, and ABCB1 expression and predicted FEV1% in COPD patients.

FIG. 10 depicts graphs showing the correlation of GCα and β expression in COPD patients and predicted FEV1% in patients with COPD.

FIG. 11 depicts graphs showing correlation of PDE4 isoform expression and lung function in COPD patients.

FIG. 12 shows the effect of CSE exposure (and rescue by RNO and/or DEX, or NAC) in expression of MKP1 and MIF, as well as phosphorylation of ERK1/2.

FIG. 13 shows the effect of CSE exposure (and rescue by RNO and/or DEX, or NAC) in expression of PI3Kδ, GCβ, and HDAC2.

FIG. 14 shows the effect of CSE exposure in expression of PDE4B and PDE4D isoforms.

FIG. 15 shows RNO dose-dependent inhibition of ROS generation in neutrophils from COPD patients after exposure to NAC.

FIG. 16 shows a reduction in glucocorticoid resistance after exposure to LY-294002 and NAC.

### DETAILED DESCRIPTION OF THE INVENTION

Unless otherwise defined, all technical and scientific terms used herein have the same meaning as commonly understood by one of ordinary skill in the art to which this invention pertains. Methods and materials similar or equivalent to those described herein can be used in the practice of the present invention, and exemplified suitable methods and materials are described below. For example, methods may be described which comprise more than two steps. In such methods, not all steps may be required to achieve a defined goal and the invention envisions the use of isolated steps to achieve these discrete goals. In addition, the materials, methods, and examples are illustrative only and not intended to be limiting.

### I. Definitions

The term “PDE4 inhibitor” means a selective phosphodiesterase inhibitor, which inhibits selectively the type 4 phosphodiesterase when compared to other known types of phosphodiesterases, e.g. type 1, 2, 5 etc., whereby the compound has a lower IC₅₀ for the type 4 phosphodiesterase by a factor of 10 compared to the IC₅₀ for the inhibition of other known phosphodiesterases, e.g. type 1, 2, 5, etc. Exemplary PDE4 inhibitors include roflumilast, rolipram, 4-(3-butoxy-4-methoxybenzyl)-2-imidazolidinone, cilomilast, arofylline, tofimilast, oglemilast and tetomilast or those disclosed in WO01/32127. In one embodiment, a PDE4 inhibitor is roflumilast.

The term “roflumilast” is represented by the chemical name, N-(3,5-dichloropyrid-4-yl)-3-cyclopropylmethoxy-4-yl)-3-cyclopropylmethoxy-4-difluoromethoxy-benzamide. As used herein, roflumilast is not limited to the free base, but also includes pharmaceutically acceptable salts of roflumilast, pharmaceutically acceptable esters of roflumilast, pharmaceutically acceptable salts of esters of roflumilast, prodrugs, pharmaceutically acceptable salts of prodrugs of roflumilast, isomers of roflumilast, and N-oxides of roflumilast. In one embodiment, roflumilast is a free base. In another embodiment, roflumilast is roflumilast N-oxide (RNO).

The term “corticosteroid” means a naturally occurring or synthetic compound characterized by a hydrogenated cyclopentanoperhydrophenanthrene ring system. As used herein, corticosteroids include pharmaceutically acceptable salts, esters, or salts of esters of corticosteroids, prodrugs of corticosteroids, and pharmaceutically acceptable salts of prodrugs of corticosteroids. Exemplary corticosteroids include class of selective glucocorticosteroid receptor agonists (SEGRAs), 11-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; Cortisol; Cortisol acetate; Cortisol butyrate; Cortisol cypionate; Cortisol octanoate; Cortisol sodium phosphate; Cortisol sodium succinate; Cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furcate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone, metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin. In one embodiment, a corticosteroid may be included in any dosage form, e.g., oral, inhaled or an injectable dosage form. In another embodiment a corticosteroid is in an oral dosage form.

The term “leukotriene receptor antagonist” means an compound which inhibits selectively the action of leukotrienes on leukotriene receptors CysLT₁ and CysLT₂. Leukotrienes constitute a group of locally acting hormones, produced in living systems from arachidonic acid. The major leukotrienes are leukotriene B₄ (LTB₄), LTC₄, LTD₄, LTE₄, and LTF₄. Biosynthesis of leukotrienes begins with the action of the enzyme 5-lipoxygenase on arachidonic acid to produce the epoxide known as leukotriene A₄ (LTA₄), which is converted to the other leukotrienes by subsequent enzymatic steps. In particular, an “LTD₄ receptor antagonist” selectively inhibits the action of leukotriene type D₄ on the cysteinyl leukotriene receptor CysLT₁ when compared to other leukotrienes, e.g. type C₄, E₄ etc., that bind to the same receptor. In one embodiment, a leukotriene receptor antagonist is montelukast. Montelukast is also known to be an LTD₄ receptor antagonist. Other leukotriene receptor antagonists (but not necessarily LTD₄ receptor antagonists) include, but are not limited to, 3(S)-[2-(carboxyethyl)thio]-3-[2-(8-phenyloctyl)phenyl]-propionic acid, N-(ethoxycarbonyl)-4-[3-[4-[1-(4-hydroxyphenyl)-1-methylethyl]-phen-oxymethyl]benzyloxy]benzenecarboximidamide, 5-[2-(2-carboxyethyl)-3-[6-(4-methoxyphenyl)-5(E) hexenyloxy]phenoxy]pentanoic acid, (2S,5S)-trans-2-(4-fluorophenoxymethyl)-5-(4-N-hydroxyureidyl-1-butynyl)-tetrahydrofuran, 4-[6-acetyl-3-[3-(4-acetyl-3-hydroxy-2-propylphenylthio)-propoxy]-2-propylphenoxy]butyric acid, (R)—N-[3-[5-(4-fluorobenzyl)thien-2-yl]-1-methyl-2-propynyl]-N-hydroxy-urea (atreleuton), 3-(1H-tetrazol-5-yl)oxanilic acid (acitazanolast), N-hydroxy-N-[1-(benzothiophen-2-yl)ethyl]urea (zileuton), cyclopentyl-3-{2-methoxy-4-[(2-methyl-phenylsulfonyl)carbamoyl]benzyl}-1-methylindol-5-carbamate (zafirlukast), 8-[4-(4-phenylbut-oxy)benzamido]-2-(tetrazol-5-yl)-4H-1-benzopyran-4-one (pranlukast), and 1-[[[(1R-1-[3-[(1E)-2-(7-chloro-2-quinolinyl)ethenyl]phenyl]-3-[2-(1-hydroxy-1-methylethyl) phenyl]propyl]thio]methyl]cyclopropaneacetic acid (montelukast), pharmaceutically acceptable salts, pharmaceutically acceptable esters, pharmaceutically acceptable salts of esters, prodrugs, and pharmaceutically acceptable salts of prodrugs thereof,

The term “montelukast” is represented by the chemical name, 1-[[[(1R-1-[3-[(1E)-2-(7-chloro-2-quinolinyl)ethenyl]phenyl]-3-[2-(1-hydroxy-1-methylethyl) phenyl]propyl]thio]methyl]cyclopropaneacetic acid. As used herein, montelukast is not limited to the free acid, but also includes pharmaceutically acceptable salts of montelukast, pharmaceutically acceptable esters of montelukast, pharmaceutically acceptable salts of esters of montelukast, prodrugs of montelukast, and pharmaceutically acceptable salts of prodrugs of montelukast, and isomers of montelukast. In one embodiment, montelukast is montelukast sodium.

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly, in humans.

The term “pharmaceutically acceptable salt” represents those salts which are suitable for use in contact with the tissues of humans and lower animals without undue toxicity, irritation, allergic response and the like, and are commensurate with a reasonable benefit/risk ratio. Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, DIPEAtes, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxyethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. In one embodiment, the pharmaceutically acceptable salt can be a hydrochloride, a hydrobromide, a hydroformate, or a maleate salt.

The term “in combination” means a pharmaceutical composition that places no limit, i.e., method, form, etc., on the administering of a compound in combination with another compound. For example, in one embodiment, a pharmaceutical composition comprises roflumilast and a corticosteroid, as discrete dosage forms e.g., one may be an oral preparation and the other may be an inhaled dose form, or as same dosage forms, or in separate containers, e.g., blisters. In another embodiment, both roflumilast and corticosteroid are administered at the same time or are taken sequentially administered about 5 minutes apart, or about 15 minutes apart, or about 30 minutes apart, or about 1 hour apart, or about 2 hours apart, or about 4 hours apart, or about 8 hours apart, or about 12 hours apart, or about 24 hours apart, wherein roflumilast is administered earlier than the corticosteroid, or vice versa. In another embodiment, roflumilast and a corticosteroid are administered together in a single dosage form, e.g., fixed dose combination.

In another embodiment, a pharmaceutical composition comprises both roflumilast and a leukotriene receptor antagonist, e.g., a LTD₄ receptor antagonist as discrete dosage forms. In yet another embodiment, both roflumilast and a leukotriene receptor antagonist are administered together in a single dosage form, e.g., fixed dose combination. In another embodiment, the roflumilast and leukotriene receptor antagonist are administered at the same time, or are taken sequentially administered about 5 minutes apart, or about 15 minutes apart, or about 30 minutes apart, or about 1 hour apart, or about 2 hours apart, or about 4 hours apart, or about 8 hours apart, or about 12 hours apart, or about 24 hours apart, wherein roflumilast is administered earlier than the leukotriene receptor antagonist, or vice versa. Other embodiments also encompass a combination comprising roflumilast, a corticosteroid, and a leukotriene receptor antagonist, as pharmaceutical compositions administered together as a single dosage form, e.g., a fixed dose combination, or as discrete dosage forms. Such combinations may also be administered concurrently or sequentially.

The term “treating or treatment” means relieving, alleviating, delaying, reducing, reversing, improving, managing and/or prevent the progress of a disease, disorder, or condition; controlling a disease, disorder, or condition; delaying the onset of a disease, disorder, or condition; ameliorating one or more symptoms characteristic of a disease, disorder, or condition; or delaying the recurrence of a disease, disorder, or condition, or characteristic symptoms thereof, depending on the nature of the disease, disorder, or condition and its characteristic symptoms.

The term “subject” means animals, including both males and females. In one embodiment, subject means mammals. In another embodiment, subject means humans.

The term “effective amount” means the amount of a formulation or composition according to the invention that, when administered to a patient for treating a state, disorder or condition is sufficient to effect such treatment. The “effective amount” will vary depending on the active ingredient, the state, symptoms, disorder, disease, or condition to be treated and its severity, and the age, weight, physical condition and responsiveness of the mammal to be treated.

The term “therapeutic effect” or “therapeutically effective” means an amount or dose of an active ingredient (e.g., PDE4 inhibitor, corticosteroid, or leukotriene receptor antagonist) that elicits the biological or medicinal response in a tissue, system, or subject that is being sought by a researcher, veterinarian, medical doctor, patient or other clinician, which includes reduction or alleviation of the symptoms of the disease being treated. As used herein, with respect to the pharmaceutical compositions described herein, the term “therapeutically effective amount/dose” refers to the amount/dose of the compound that, when combined, is sufficient to produce an effective response upon administration to a patient.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per usual or customary practice in the art. Alternatively, “about” with respect to amounts can mean plus or minus a range of up to 20%, preferably up to 10%, more preferably up to 5%.

### II. Pharmaceutical Compositions

In one embodiment, the present invention provides a pharmaceutical composition comprising roflumilast in combination with a corticosteroid and/or a leukotriene receptor antagonist, each intermixed with a pharmaceutically acceptable carrier. In one embodiment, the pharmaceutically acceptable carriers are present in two or more discrete dosage forms, each dosage form having an active ingredient of the combination. In another embodiment, the active ingredients of the combination are intermixed in a single dosage form. In yet another embodiment, the dosage form is intended for oral use.

Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc.

In another embodiment, the present invention provides a pharmaceutical composition comprising roflumilast or a pharmaceutically acceptable salt, ester, prodrug, or pharmaceutically acceptable salts of esters or prodrugs, or N-oxide thereof in combination with a corticosteroid wherein the corticosteroid is selected from the group consisting of dexamethasone, prednisone and budesonide, or a pharmaceutically acceptable salt, ester, prodrug or pharmaceutically acceptable salt of an ester or a prodrug thereof, and optionally together with, or in an alternative combination with a leukotriene receptor antagonist, wherein the leukotriene receptor antagonist is montelukast, or a pharmaceutically acceptable salt, ester, prodrug, or pharmaceutically acceptable salt of an ester or prodrug thereof.

In another embodiment, the present invention provides a pharmaceutical composition comprising roflumilast or a pharmaceutically acceptable salt, ester, prodrug, or pharmaceutically acceptable salts of esters or prodrugs, or N-oxide thereof in combination with dexamethasone or a pharmaceutically acceptable salt, ester, prodrug or pharmaceutically acceptable salt of an ester or prodrug thereof.

In another embodiment, the present invention provides a pharmaceutical composition comprising roflumilast or a pharmaceutically acceptable salt, ester, prodrug, or pharmaceutically acceptable salt of an ester or prodrug thereof or N-oxide thereof in combination with prednisone or a pharmaceutically acceptable salt ester, prodrug or pharmaceutically acceptable salt of an ester or prodrug thereof.

In another embodiment, the present invention provides a pharmaceutical composition comprising roflumilast or a pharmaceutically acceptable salt, ester, prodrug, or pharmaceutically acceptable salts of esters or prodrugs or N-oxide thereof in combination with budesonide or a pharmaceutically acceptable salt, prodrug or pharmaceutically acceptable salt of an ester or prodrug thereof.

In other embodiments, the present invention provides a pharmaceutical composition comprising roflumilast or a pharmaceutically acceptable salt, ester, prodrug,

or pharmaceutically acceptable salts of esters or prodrugs, or N-oxide thereof in combination with a leukotriene receptor antagonist or a pharmaceutically acceptable salt, ester, prodrug, or pharmaceutically acceptable salt of an ester or prodrug thereof.

In another embodiment, the present invention provides a pharmaceutical composition comprising roflumilast or a pharmaceutically acceptable salt, ester, prodrug, or pharmaceutically acceptable salts of esters or prodrugs, or N-oxide thereof in combination with montelukast or a pharmaceutically acceptable salt, ester, prodrug, or pharmaceutically acceptable salt of an ester or prodrug thereof.

We have surprisingly found that the administration of roflumilast in combination with a corticosteroid is advantageous in the context of restoring corticoid sensitivity in inflammatory cells activated in COPD. In one embodiment, the present invention provides for small doses of either roflumilast or corticosteroid or both, wherein the small doses are such that they are less than the optimum dose of either PDE4 inhibitor or corticosteroid for a therapeutic effect. In another embodiment, small doses of either active ingredient of the present invention are administered simultaneously or sequentially.

The present invention also provides for administration of roflumilast in combination with a leukotriene receptor antagonist, e.g., montelukast, for treatment of COPD. In one embodiment, small doses of either roflumilast or leukotriene receptor antagonist, or both, are administered such that the small doses are in amounts less than the optimum dose of either PDE4 inhibitor or leukotriene receptor antagonist what is needed to observe a therapeutic effect. In other embodiments, small doses of the PDE4 inhibitor or the leukotriene receptor antagonist are administered simultaneously or sequentially.

### III. Dosage Forms

In one embodiment, the present invention provides for an oral dosage form administered as discrete solid units, e.g., capsules, tablets, pills, powders, granules etc. Where the oral dosage form is in the form of a tablet, any pharmaceutical carrier, diluents (such as sucrose, mannitol, lactose, starches) or excipients known in the art, including but not limited to suspending agents, solubilizers, buffering agents, binders, disintegrants, preservatives, colorants, flavorants, lubricants may be used. In another embodiment, the present invention provides an oral dosage form for one of or each of PDE4 inhibitor and corticosteroid and/or leukotriene receptor antagonist.

In another embodiment, the present invention provides for an oral dosage administered as a liquid form. Exemplary liquid oral dosage forms are aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention.

In yet another embodiment, the present invention provides for an injectable dosage form, for example, intravenously, in the form of an isotonic sterile solution. In yet another embodiment, the present invention provides an injectable dosage form for either or both of PDE4 inhibitor and corticosteroid.

In another embodiment, the present invention provides for an inhalable dosage form, for example in the form of a powder (e.g., micronized) or in the form of atomized solutions or suspensions. In yet another embodiment, the present invention provides an inhalable dosage form for one of or each of PDE4 inhibitor and corticosteroid and/or leukotriene receptor antagonist.

### IV. Dosage Quantities

The dosage of the pharmaceutical composition of the present invention will vary depending on the symptoms, the treatment desired, age and body weight of the subject, the nature and severity of the disorder to be treated, the route of administration and pharmacokinetics of the active ingredients. The frequency of the dose indicated will also vary with the treatment desired and the disorder indicated.

In one embodiment, a PDE4 inhibitor is administered in combination with a corticosteroid and/or a leukotriene receptor antagonist in an amount sufficient to achieve a therapeutic effect. The dosage range for a PDE4 inhibitor ranges from about 0.01 μg to about 100 mg per day. In another embodiment, amount for a PDE4 inhibitor ranges from about 0.01 μg to about 0.025 μg per day, or about 0.025 μg to about 0.05 μg per day, or about 0.05 μg to about 1 μg per day, or about 1 μg to about 10 μg per day, or about 10 μg to about 100 μg per day, or about 100 μg to about 500 μg per day, or about 500 μg to about 750 μg per day, or about 750 μg to about 1.5 mg per day or about 1.5 mg to about 5 mg per day, or about 5 mg to about 100 mg per day.

The dosage range for a corticosteroid ranges from about 0.01 μg to about 100 mg per day. In another embodiment, amount for a corticosteroid ranges from about 0.01 μg to about 0.025 μg per day, or about 0.025 μg to about 0.05 μg per day, or about 0.05 μg to about 1 μg per day, or about 1 μg to about 10 μg per day, or about 10 μg to about 100 μg per day, or about 100 μg to about 500 μg per day, or about 500 μg to about 750 μg per day, or about 750 μg to about 1.5 mg per day or about 1.5 mg to about 5 mg per day, or about 5 mg to about 100 mg per day.

The dosage range for a leukotriene receptor antagonist (such as an LTD₄ receptor antagonist) is about 0.001 mg to about 100 mg/kg body weight, preferably 0.01 mg to about 10 mg/kg, and most preferably 0.1 to 1 mg/kg, in single or divided doses. In some instances, it may be necessary to use dosages outside the aforementioned ranges.

For intravenous administration, a suitable dosage range for a leukotriene receptor antagonist is from about 0.001 mg to about 25 mg (preferably from 0.01 mg to about 1 mg) per kg of body weight per day. Where an oral composition is administered, a suitable dosage range can be, e.g. from about 0.01 mg to about 100 mg of a leukotriene receptor antagonist per kg of body weight per day, preferably from about 0.1 mg to about 10 mg per kg.

In yet another embodiment, the dosage of roflumilast is about 0.05 mg/kg of body weight per day, or about 0.1 mg/kg of body weight per day, or about 0.3 mg/kg of body weight per day, or about 1 mg/kg of body weight per day or about 5 mg/kg of body weight per day; the dosage for corticosteroid wherein the corticosteroid is selected from the group consisting of dexamethasone, prednisone and budesonide is about 0.05 mg/kg of body weight per day, or about 0.1 mg/kg of body weight per day, or about 0.3 mg/kg of body weight per day, or about 1 mg/kg of body weight per day or about 5 mg/kg of body weight per day; and the dosage for leukotriene receptor antagonist wherein the leukotriene receptor antagonist is montelukast is about 0.01 mg/kg of body weight per day, or about 0.1 mg/kg of body weight per day, or about 1 mg/kg of body weight per day or about 5 mg/kg of body weight per day. One skilled in the art will appreciate that the administered doses can be converted to a human equivalent dose per the FDA guidance document titled “FDA Guidance for Industry, Estimating the Maximum Safe Starting Dose in Initial Clinical Trials for Therapeutics in Adult Healthy Volunteers, July 2005.

Roflumilast is known to one skilled in the art. In one embodiment, the recommended dose of roflumilast is 500 μg per day administered orally. In other embodiments, the dose of roflumilast may range from 50 μg up to 500 μg, in single or divided doses. In another embodiment, where a leukotriene receptor antagonist like montelukast is co-administered, the recommended dose is 4 to 10 mg per day

### V. Methods of Treatment

In one embodiment, the present invention provides for method of treating COPD by administering to a subject a pharmaceutical composition comprising a PDE4 inhibitor in combination with a corticosteroid according to any of the embodiments described in the foregoing sections.

In another embodiment, the present invention provides for a method of treating COPD by administering to a subject a pharmaceutical composition comprising roflumilast in combination with a corticosteroid.

In another embodiment, the present invention provides for a method of treating COPD by administering to a subject a pharmaceutical composition comprising roflumilast or a pharmaceutically acceptable salt, ester, prodrug, or pharmaceutically acceptable salts of esters or prodrugs thereof, or N-oxide thereof in combination with a corticosteroid wherein the corticosteroid is selected from the group consisting of dexamethasone, prednisone and budesonide, or a pharmaceutically acceptable salt, ester, prodrug or pharmaceutically acceptable salt of an ester or prodrug thereof.

In another embodiment, the present invention provides for a method of treating COPD by administering to a subject a pharmaceutical composition comprising roflumilast or a pharmaceutically acceptable salt, ester, prodrug, or pharmaceutically acceptable salts of esters or prodrugs thereof, or N-oxide thereof in combination with dexamethasone or a pharmaceutically acceptable salt, ester, prodrug or pharmaceutically acceptable salt of an ester or prodrug thereof.

In another embodiment, the present invention provides for a method of treating COPD by administering to a subject a pharmaceutical composition comprising roflumilast or a pharmaceutically acceptable salt, ester, prodrug, or pharmaceutically acceptable salts of esters or prodrugs thereof, or N-oxide thereof in combination with prednisone or a pharmaceutically acceptable salt, ester, prodrug or pharmaceutically acceptable salt of an ester or prodrug thereof.

In another embodiment, the present invention provides for a method of treating COPD by administering to a subject a pharmaceutical composition comprising roflumilast or a pharmaceutically acceptable salt, ester, prodrug, or pharmaceutically acceptable salts of esters or prodrugs thereof, or N-oxide thereof in combination with budesonide or a pharmaceutically acceptable salt, ester, prodrug or pharmaceutically acceptable salt of an ester or prodrug thereof.

In another embodiment, the present invention provides a method to enhance the sensitivity of corticosteroid in treating an inflammatory response associated with COPD by administering to a subject a PDE4 inhibitor in combination with a corticosteroid.

In yet another embodiment, the treatment of COPD involves administering smaller doses which are less than the optimum dose of either PDE4 inhibitor or corticosteroid, or both until a therapeutic effect is attained. In smaller doses, either PDE4 inhibitor or corticosteroid will offer negligible therapeutic benefits when administered alone. In yet another embodiment, the PDE4 inhibitor in combination with a corticosteroid is administered simultaneously or sequentially to attain the necessary therapeutic effect.

In another embodiment, the present invention provides for method of treating COPD by administering to a subject a pharmaceutical composition comprising a PDE4 inhibitor in combination with a leukotriene receptor antagonist according to any of the embodiments described in the foregoing sections.

In another embodiment, the present invention provides for a method of treating COPD by administering to a subject a pharmaceutical composition comprising roflumilast in combination with a leukotriene receptor antagonist.

In another embodiment, the present invention provides for a method of treating COPD by administering to a subject a pharmaceutical composition comprising roflumilast or a pharmaceutically acceptable salt, ester, prodrug, or pharmaceutically acceptable salts of esters or prodrugs thereof, or N-oxide thereof in combination with montelukast, or a pharmaceutically acceptable salt, ester, prodrug or pharmaceutically acceptable salt of an ester or prodrug thereof.

The invention will now be described in greater detail by reference to the following non-limiting examples.

### EXAMPLES 1 TO 7

### Pharmacology

The efficacy of PDE4 inhibitors combined with corticosteroids was examined on different functional and mechanistic outputs, e.g., secretion and expression of inflammatory mediators resistant to corticosteroids, or enzymes, or transcription factors in various in-vitro models, e.g., oxidative stress, inflammation etc., relevant to the pathogenic mechanisms of COPD.

Peripheral neutrophils and monocytes as well as whole blood were obtained from COPD and healthy non-smoker volunteers. Clinical characteristics of patients are defined in Table 1. Thirty-two patients with COPD, defined according to GOLD guidelines, were enrolled in this study. Patients were aged 68.7±10 years, FEV1 67.6±18% predicted, and 15 were prescribed an inhaled corticosteroid. All patients were current smokers. There were no exacerbations of the disease within 2 weeks prior to taking blood samples.

Twenty-five age-matched non-smoking control subjects with normal lung function (age 65±4 years old, FEV1 98±3% predicted) who did not have any respiratory disease, were also recruited as normal controls, respectively. Routine lung function tests were performed to evaluate forced vital capacity (FVC), forced expiratory volume in 1 s (FEV1) and FEV1/FVC ratio using a Vitalograph® alpha III spirometer (Vitalograph, Maids Moreton, UK).

TABLE 1
Clinical features. COPD: chronic obstructive pulmonary
disease; FEV1: forced expiratory volume in one second;
FVC: forced vital capacity; Pack-yr = 1 year smoking
20 cigarettes-day. Data are mean ± SE.

| | Healthy | COPD |
|---|---|---|
| | (n = 25) | (n = 32) |
| Age, yr | 65 ± 4 | 68,7 ± 10 |
| Sex (M/F) | 15/10 | 21/11 |
| Tobacco consumption, pack-yr | 0 | 32,5 ± 15 |
| FEV1, % pred | 98 ± 3 | 67.6 ± 18 |
| FVC, % pred | 89 ± 6 | 92.5 ± 14 |
| FEV1/FVC % | 94 ± 5 | 69.2 ± 11 |

### Human Neutrophil and Monocyte Isolation

Neutrophils and monocytes were isolated from peripheral venous blood by standard laboratory procedures. In brief, peripheral venous blood was mixed with dextran 500 at 3% (in 0.9% saline) in a proportion of 2:1. This mixture was incubated at room temperature for 30 min until erythrocytes were sedimented. The upper phase was carefully collected and added on Ficoll-Paque Histopaque 1077 (Amershan Pharmacia Biotech, Barcelona, Spain) density gradient in a proportion of 3:1. The two phases generated were centrifuged at 150 g, 4° C. for 30 min. Thus, the interface was collected to isolate monocytes, and the pellet obtained (which is consisted a mixture of neutrophils and low proportion of residual erythrocytes and traces of eosinophils and basophils) was resuspended in an erythrocyte lysis buffer (Biolegend, UK) for 5 min in ice. Cell suspension was washed two times with phosphate buffer (PBS).

To isolate monocytes from the interface, the interface cell suspension was adjusted to 500×10³ cells per well in 24-well plates and incubated for 4 h before non-adherent cells were discarded and remaining cells were kept in RPMI containing 0.25% FCS for at least 6 h before stimulation.

The preparations were >97% pure in neutrophils and >96% in monocytes, as assessed by Giemsa staining, and had a viability of >99%, measured by trypan blue exclusion. Neither purity nor viability was affected in the study's different experimental conditions.

### Preparation of Cigarette Smoke Extract Solutions

CSE was prepared as previously outlined (Milara et al.; Ortiz et al.). Briefly, the smoke of a research cigarette (2R4F; Tobacco Health Research, University of Kentucky, Ky., USA) was generated by a respiratory pump (Apparatus Rodent Respirator 680; Harvard, Germany) through a puffing mechanism related to the human smoking pattern (3 puff/min; 1 puff 35 ml; each puff of 2 s duration with 0.5 cm above the filter) and was bubbled into a flask containing 25 ml of pre-warmed (37° C.) RPMI 1640 culture medium supplemented as describe above. The CSE solution was sterilized by filtration through a 0.22-μm cellulose acetate sterilizing system (Corning, N.Y.). The resultant CSE solution was considered to be 100% CSE and was used for experiments within 30 min of preparation. CSE 10% corresponds approximately to the exposure associated with smoking two packs per day (Su et al., 1998). The quality of the prepared CSE solution was assessed based on the absorbance at 320 nm, which is the specific absorption wavelength of peroxynitrite. Stock solutions with an absorbance value of 3.0±0.1 were used. To test for cytotoxicity from CSE, isolated neutrophils and monocytes were treated with CSE concentrations of up to 5% for 24. No significant difference in the lactate dehydrogenase supernatant level (lactate dehydrogenase cytotoxicity assay; Cayman, Spain) was observed in comparison with the control group.

### IL-8, MMP-9, IL-1β, GM-CSF and CCL-5 Measurement

Peripheral neutrophil cell suspension was adjusted to 500×10³ cells per well in 24-well plates and incubated in RPMI culture medium for 1 h into the incubator at 37° C., 5% CO₂. Cells were then treated in presence or absence of RNO (0.1 nM-1 μM), DEX (0.1 nM-1 μM), the antioxidant N-acetyl-1-cysteine (1 mM) or the PI3K pan-inhibitor LY-294002 (10 μM) for 1 h before the stimulation with LPS (1 μg/ml) or CSE 5%. Both the stimulus and drug were remained together for 6 h. Supernatants were collected and centrifuged at 120 g for 5 min, and the free-cell supernatant was used to measure IL-8, MMP-9, IL-1β, GM-CSF and CCL-5.

IL-8 was determined by using commercially available enzyme-linked immunosorbent assay kit for IL-8 (R&D Systems, UK) according to the manufacturer's protocol. MMP-9, IL-1β, GM-CSF and CCL-5 were measured by LUMINEX technology according to the manufacturer's protocol.

### Real Time RT-PCR

Total RNA was obtained from isolated neutrophils under basal conditions or after different drug and stimulation periods defined above by using TriPure Isolation Reagent (Roche, Indianapolis, USA). Integrity of the extracted RNA was confirmed with Bioanalyzer (Agilent, Palo Alto, Calif., USA). The reverse transcription was performed in 300 ng of total RNA with the TaqMan reverse transcription reagents kit (Applied Biosystems, Perkin-Elmer Corporation, CA, USA). cDNA was amplified using assays-on-demand specific primers pre-designed by Applied Biosystems for MIF, GCα, GCβ, MKP1, PI3K-δ, HDAC2, P-glycoprotein and PDE4 A, B, C and D isoform genes in a 7900HT Fast Real-Time PCR System (Applied Biosystems) using Universal Master Mix (Applied Biosystems). Relative quantification of these transcripts was determined with the 2^{-ΔΔCt} method using glyceraldehyde phosphate dehydrogenase (GAPDH) as endogenous control (Applied Biosystems; 4352339E) and normalized to control group as previously described (Milara et al., 2009).

### Western Blot of ERK1/2

Western blot analysis was used to detect changes in p-ERK1/2 (42-44 kD). Neutrophils were incubated in RPMI basal culture medium and treated with RNO, DEX, their combination, or with the antioxidant NAC for 1 h and stimulated with CSE 5% for 20 min. Cells were then centrifuged and the total protein was extracted using a lysis buffer consisting of a complete inhibitor cocktail plus 1 mM ethylenediaminetetraacectic acid (Roche Diagnostics Ltd, West Sussex, UK) with 20 mM Tris base, 0.9% NaCl, 0.1% Triton X-100, 1 mM dithiothreitol and 1 μg mL⁻¹ pepstatin A. The Bio-Rad assay (Bio-Rad Laboratories Ltd., Herts, UK) was used (following manufacturer's instructions) to quantify the level of protein in each sample to ensure equal protein loading. Sodium dodecyl sulfate polyacrylamide gel electrophoresis was used to separate the proteins according to their molecular weight. Briefly, 20 μg proteins (denatured) along with a molecular weight protein marker, Bio-Rad Kaleidoscope marker (Bio-Rad Laboratories), were loaded onto an acrylamide gel consisting of a 5% acrylamide stacking gel stacked on top of a 10% acrylamide resolving gel and run through the gel by application of 100 V for 1 h. Proteins were transferred from the gel to a polyvinylidene difluoride membrane using a wet blotting method. The membrane was blocked with 5% Marvel in PBS containing 0.1% Tween20 (PBS-T) and then probed with a rabbit anti-human p-ERK1/2 (1:1,000) antibody (monoclonal antibody; Cell Signalling, Boston, Mass., USA; catalogue no. 4376S) and normalized to total rabbit anti-human ERK1/2 (1:1,000) antibody (monoclonal antibody; Cell Signalling, Boston, Mass., USA; catalogue no. 4695). The enhanced chemiluminescence method of protein detection using enhanced chemiluminescence reagents, ECL plus (Amersham GE Healthcare, Buckinghamshire, UK), was used to detect labelled proteins.

### Nuclear Protein Extraction and Quantification

Nuclear protein extraction was performed to measure total HDAC activity with the active motif extraction kit (Active Motif Europe, Rixensart, Belgium) in a total of 8×10⁶ cells per condition according to the manufacturer's protocol. The Bio-Rad assay (Bio-Rad Laboratories Ltd., Herts, UK) was adopted (following manufacturer's instructions) to quantify the level of protein in each sample to ensure equal nuclear protein loading to measure HDAC activity.

### HDAC Activity

To measure HDAC activity, neutrophils from COPD patients were incubated in presence or absence of RNO, DEX or NAC for 1 h and stimulated with CSE 5% for another 1 h. Cells were then washed and centrifuged to extract the nuclear protein.

HDAC activity was measured by use of a colorimetric assay system (AK-501, Biomol) according to the manufacturer's protocol. Essentially, the procedure involves the use of HDAC colorimetric substrate (Color de Lys substrate, 500 μM), which comprises an acetylated lysine side chain and is incubated with a sample containing nuclear extract. Deacetylation sensitizes the substrate and treatment with the lysine developer produces a chromophore which can be analyzed with a colorimetric plate reader at 405 nm. HeLa cell nuclear extract was used as a positive control. A standard curve was prepared, using the known amount of the deacetylated standard (Boc-Lys-pNA) included in the kit. Results were expressed as μM of HDAC per mg of nuclear protein.

### Measure of Intracellular Reactive Oxygen Species (ROS)

Intracellular reactive oxygen species (ROS) were measured by a flow cytometry assay using 2′,7′-dichlorodihydrofluorescein diacetate (H₂DCF-DA, Molecular Probes, UK) as previously outlined (Milara et al., 2010). H₂DCF-DA was added for 15 min before stimulus to a final concentration of 5 μM to measure intracellular ROS. The bacterial peptide fMLP was added as stimulus at 1 μM for 30 min to generate intracellular ROS (Elbim et al., 2001). In other experiments, to reproduce in vivo conditions, CSE 5% was added to generate intracellular ROS. Intracellular ROS was measured using fresh whole blood from COPD patients. Blood neutrophils were selected using CD16-Alexa Fluor 647 antibody (molecular probes, UK) as neutrophil marker, and CD14-PerCEP-Cy5.5 mouse antihuman (molecular probes, UK) as monocyte marker in an Epics Profile II flow cytometer. A total of 1 ml of whole blood was pre-incubated at 37° C. for 30 min with RNO (1 nM-1 NAC (1 mM) and H₂DCF-DA followed by fMLP 1 μM or CSE 5% stimulation for another 1 h. Then erythrocytes were lysed using a commercial erythrocyte lysis buffer (Biolegend, UK) for 10 min. Fluorescence intensity was measured in the polymorphonuclear region of whole blood in an Epics Profile II flow cytometer. Results were expressed as DCF fluorescence in relative fluorescence units (RFU).

### PI3K Activity

To measure PI3K activity, neutrophils from COPD patients were isolated and incubated with RNO (1 nM, 1 μM), DEX (1 μM), the combination of RNO 1 nM plus DEX 1 μM, or with the antioxidant NAC (1 mM) for 1 h. Then cells were stimulated with CSE 5% for 30 min. After cell stimulation, neutrophils were centrifuges and total protein was extracted from neutrophils. Total protein amount was measured using the Bio-Rad assay (Bio-Rad Laboratories Ltd., Herts, UK), and PI3K activity was measured using the PI3-Kinase Activity ELISA: Pico (Catalog No. k-1000s according to the manufacturer's protocol. In brief, PI3K reactions were run with the Class I PI3K physiological substrate P1(4,5)P2 (PIP2). The enzyme reactions, PIP3 standards, and controls were then mixed and incubated with PIP3 binding protein that is highly specific and sensitive to PIP3. This mixture was then transferred to a PIP3-coated microplate for competitive binding. Afterwards, a peroxidase-linked secondary detector and colorimetric detection was used to detect the amount of PIP3 produced by PI3K through comparing the enzyme reactions with a PIP3 standard curve.

### Data Analysis

Data were presented as mean±SEM. IC₅₀ values were calculated for each drug and their combinations. Statistical analysis of results was carried out by analysis of variance (ANOVA) followed by Bonferroni test, by Student's t test, or by non-parametric tests as appropriate (GraphPad Software Inc, San Diego, Calif., USA). Significance was accepted when P<0.05. For clinical correlations of lung function and gene expression from neutrophils the non-parametric Spearman correlation analysis was carried out.

### EXAMPLE 1

### Results (IL-8 Release)

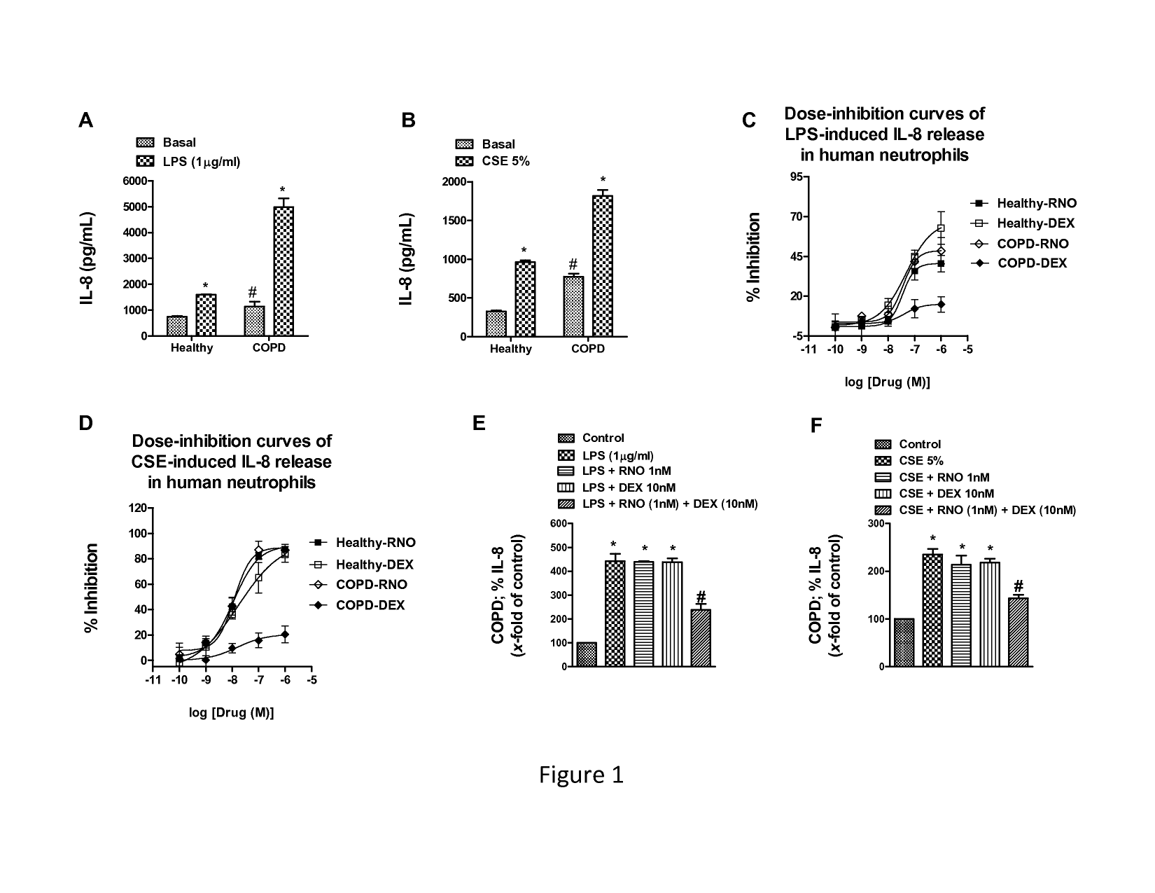

Neutrophils isolated from COPD patients showed a higher basal IL-8 release than that of healthy volunteers (FIGS. 1A and 1B; p<0.05). The bacterial endotoxin LPS (1 μg/ml) elicited an increase of IL-8 release that was significantly higher in COPD patients vs. healthy volunteers (FIG. 1A; p<0.05). CSE 5% stimulation also induced a higher IL-8 release in neutrophils from COPD patients (FIG. 1B; p<0.05). Neutrophils from healthy and COPD patients were stimulated with LPS (1 μg/ml) or CSE 5% in presence of RNO (0.1 nM-1 μM) or DEX (0.1 nM-1 μM) for 6 h and IL-8 supernatants were measured (FIGS. 1C and 1D). The −log IC₅₀ and % of maximum inhibitory effect generated are defined in Table 2. Neutrophil IL-8 release from COPD patients was resistant to DEX, which reached a maximal % inhibition of 15% and 20.6% in response to LPS or CSE stimulus.

TABLE 2
Inhibition of IL-8 release in isolated peripheral blood neutrophils from healthy
and COPD patients Inhibitory concentration-dependent curves were generated by
incubation with Roflumilast N-oxide (RNO; 0.1 nM-1 μM) or Dexamethasone
(DEX; 0.1 nM-1 μM) in response to LPS (1 μg/ml) or cigarette smoke extract
(CSE 5%). Values are mean ± SEM of 3 independent experiments run in triplicate.
IC₅₀ values for half-maximum inhibition were calculated by nonlinear
regression analysis.

| | HEALTHY | | | COPD | | |
|---|---|---|---|---|---|---|
| | Maximal | | | Maximal | | |
| Stimulus | % Inhibition | −log IC₅₀ | N | % Inhibition | −log IC₅₀ | N |
| LPS | | | | | | |
| RNO | 40.50 ± 4.55 | 7.46 ± 0.26 | 3 | 48.70 ± 5.18 | 7.44 ± 0.24 | 3 |
| DEX | 67.08 ± 11.41 | 7.32 ± 0.29 | 3 | 15 ± 4.4* | 7.37 ± 4.4 | 3 |
| CSE | | | | | | |
| RNO | 90.4 ± 5.4 | 7.95 ± 0.12 | 3 | 88.9 ± 5.62 | 7.92 ± 0.1 | 3 |
| DEX | 94.33 ± 21.7 | 7.77 ± 0.38 | 3 | 20.6 ± 12.1* | 7.81 ± 1.14 | 3 |

*p < 0.05 vs. healthy values.

In other experiments performed in neutrophils from COPD patients, the combination of RNO (1 nM) and DEX (10 nM) showed a synergistic effect on the inhibition of IL-8 release in response to LPS or CSE (FIGS. 1E and 1F).

### EXAMPLE 2

### Results (MMP-9 Release)

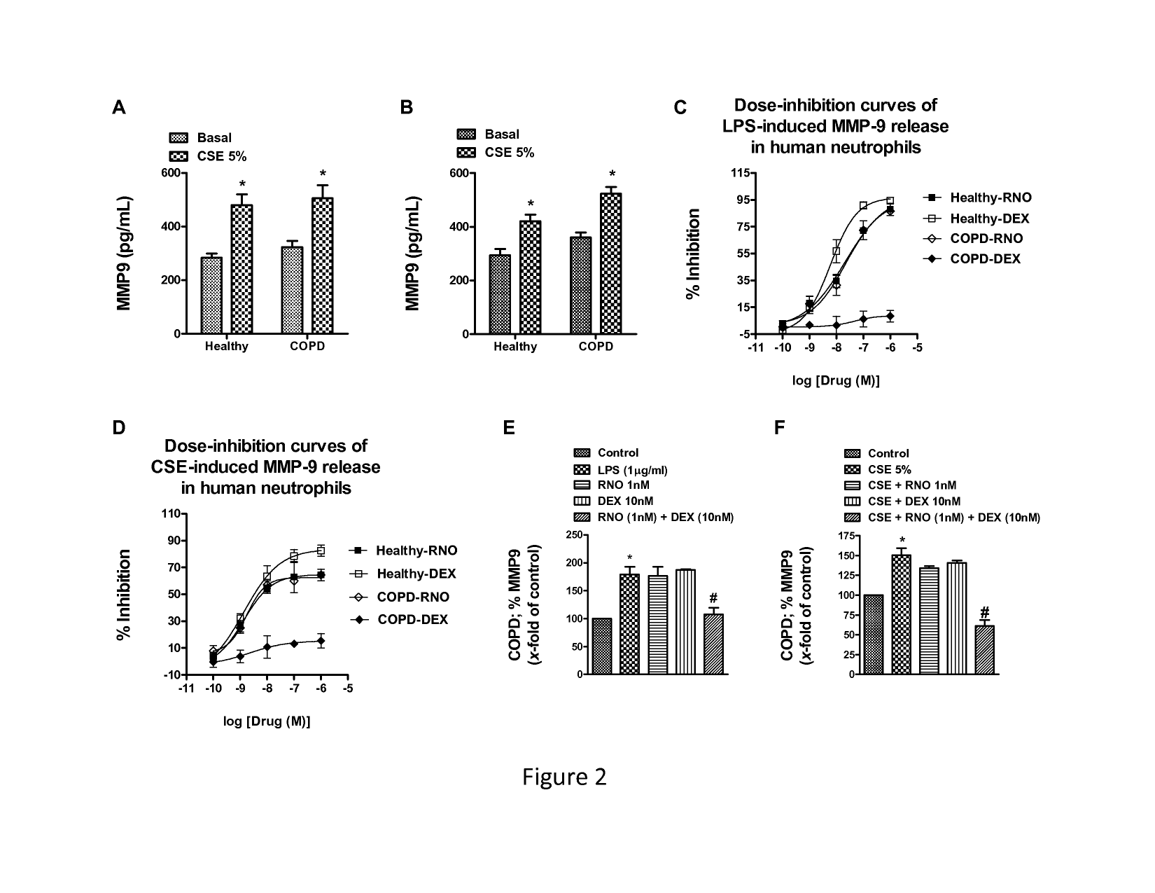

Neutrophils isolated from COPD patients showed a higher basal MMP-9 release than that of healthy volunteers although these differences were not significant (FIGS. 2A and 2B). The bacterial endotoxin LPS (1 μg/ml) as well as the CSE 5% elicited a significant increase of MMP-9 release in both COPD patients and healthy volunteers (FIGS. 2A and 2B). Neutrophils from healthy volunteers and COPD patients were stimulated with LPS (1 μg/ml) or CSE 5% in presence of RNO (0.1 nM-1 μM) or DEX (0.1 nM-1 μM) for 6 h and MMP-9 supernatants were measured. The −log IC₅₀ and % of maximum inhibitory effect generated are defined in table 3. Concentration dependent inhibitory curves are shown in FIGS. 2C and 2D. Neutrophil IL-8 release from COPD patients was resistant to DEX, which reached a maximal % inhibition of 8.8% and 15.4% in response to LPS or CSE stimulus (Table 3 and FIGS. 2C and 2D).

TABLE 3
Inhibition of MMP-9 release in isolated peripheral blood neutrophils from
healthy and COPD patients. Inhibitory concentration-dependent curves were
generated by incubation with Roflumilast N-oxide (RNO; 0.1 nM-1 μM) or
Dexamethasone (DEX; 0.1 nM-1 μM) in response to LPS (1 μg/ml) or cigarette
smoke extract (CSE 5%). Values are mean ± SEM of 3 independent
experiments run in triplicate. IC₅₀ values for half-maximum inhibition
were calculated by nonlinear regression analysis.

| | HEALTHY | | | COPD | | |
|---|---|---|---|---|---|---|
| | Maximal | | | Maximal | | |
| Stimulus | % Inhibition | −log IC₅₀ | N | % Inhibition | −log IC₅₀ | N |
| LPS | | | | | | |
| RNO | 98.9 ± 12.6 | 7.66 ± 0.22 | 3 | 94.61 ± 11.82 | 7.64 ± 0.23 | 3 |
| DEX | 96.8 ± 5.1 | 8.22 ± 0.13 | 3 | 8.8 ± 2.45* | 7.33 ± 0.49* | 3 |
| CSE | | | | | | |
| RNO | 64.8 ± 5.64 | 8.87 ± 5.6 | 3 | 62.58 ± 2.28 | 8.76 ± 0.11 | 3 |
| DEX | 83.9 ± 5.79 | 8.86 ± 5.8 | 3 | 15.44 ± 6.95* | 8.66 ± 1.6 | 3 |

*p < 0.05 vs. healthy values.

In other experiments performed in neutrophils from COPD patients, the combination of RNO (1 nM) and DEX (10 nM) showed a synergistic effect on the inhibition of MMP-9 release in response to LPS or CSE (FIGS. 2E and 2F).

### EXAMPLE 3

### Results (IL-1β Release)

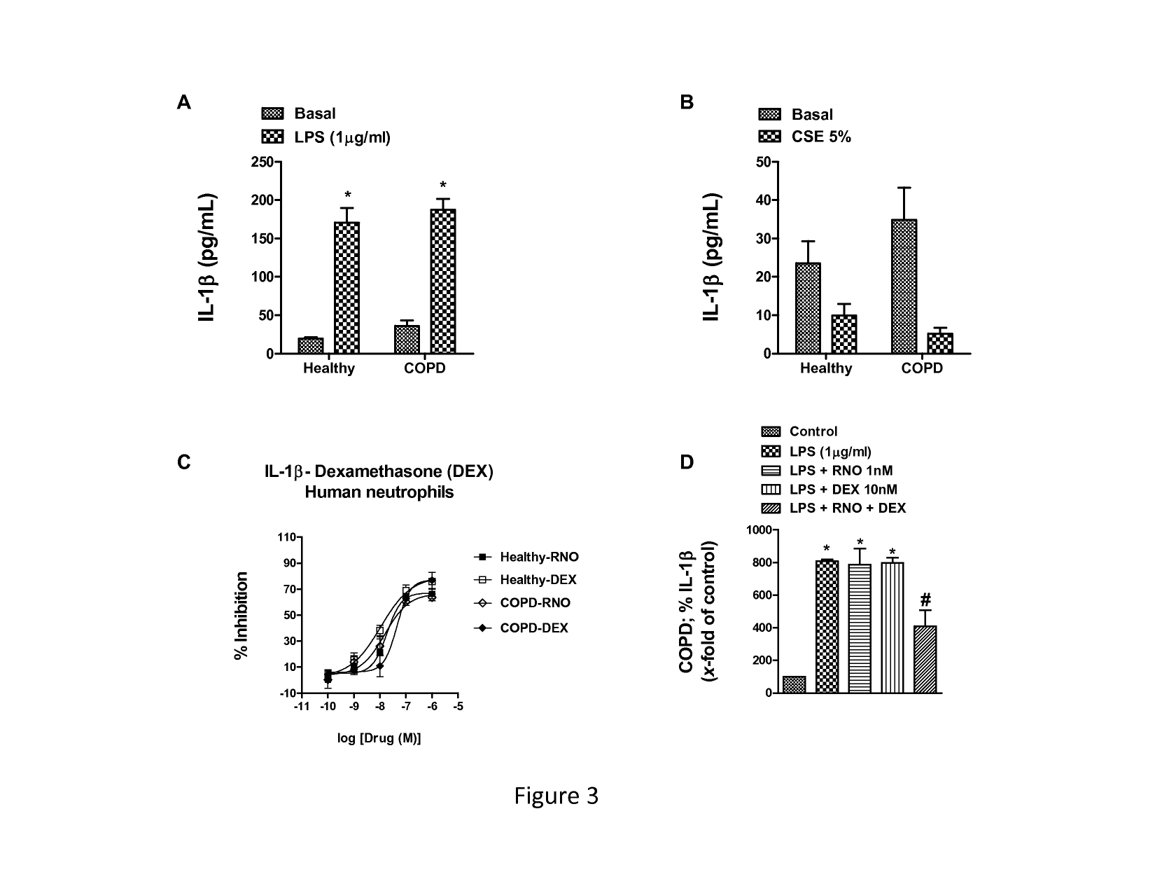

Neutrophils isolated from COPD patients did not show significant differences in basal IL-1β release when compared to neutrophils from healthy volunteers (FIGS. 3A and 3B). Notably, the bacterial endotoxin LPS (1 μg/ml) increased IL-1β release significantly (FIG. 3A), while CSE 5% decreased IL-1β release in both, COPD and healthy volunteers (FIG. 3B).

Neutrophils from healthy and COPD patients were stimulated with LPS (1 μg/ml) in presence of RNO (0.1 nM-1 μM) or DEX (0.1 nM-1 μM) for 6 h and IL-1β supernatants were measured. The −log IC₅₀ and % of the maximum inhibitory effect generated are defined in table 4. Concentration dependent inhibitory curves are shown in FIG. 3C.

Neutrophil IL-1β release from COPD patients was resistant to DEX, which reached a −log IC₅₀ of 7.34 M vs 7.97 M in healthy volunteers (Table 4 and FIG. 3C; p<0.05).

TABLE 4
Inhibition of IL-1β release in isolated peripheral blood neutrophils
from healthy and COPD patients. Inhibitory concentration-dependent
curves were generated by incubation with roflumilast N-oxide (RNO;
0.1 nM-1 μM) or dexamethasone (DEX; 0.1 nM-1 μM) in response
to LPS (1 μg/ml). Values are mean ± SEM of 3 independent
experiments that were run in triplicate. IC₅₀ values for half-
maximum inhibition were calculated by nonlinear regression analysis.

| Stim- | HEALTHY | | | COPD | | |
|---|---|---|---|---|---|---|
| ulus | Maximal | | | Maximal | | |
| LPS | % Inhibition | −log IC₅₀ | N | % Inhibition | −log IC₅₀ | N |
| RNO | 67.08 ± 6.63 | 7.73 ± 0.22 | 3 | 66.69 ± 6.86 | 7.82 ± 0.21 | 3 |
| DEX | 79.58 ± 5.57 | 7.97 ± 0.15 | 3 | 77.16 ± 5.69 | 7.34 ± 0.16* | 3 |

*p < 0.05 vs. healthy values.

In other experiments performed in neutrophils from COPD patients, the combination of RNO (1 nM) and DEX (10 nM) showed an additive effect on the inhibition of IL-1β release in response to LPS (FIG. 3D).

### EXAMPLE 4

### Results (GM-CSF Release)

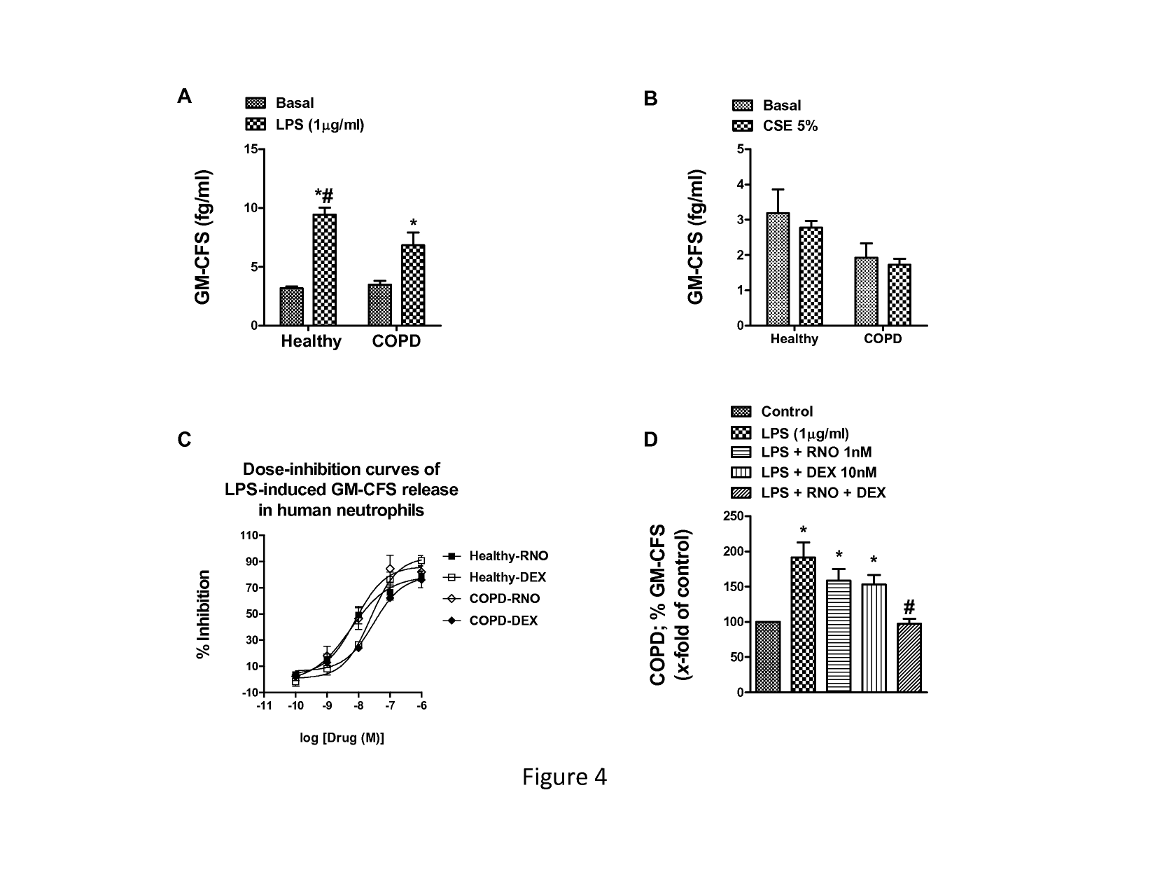

Neutrophils isolated from COPD patients did not show significant differences in basal GM-CSF release in comparison with neutrophils isolated from healthy volunteers (FIGS. 4A and 4B). The bacterial endotoxin LPS (1 μg/ml) caused an increase in GM-CSF release (FIG. 4A) while CSE 5% slightly decreased GM-CSF release in both, COPD patients and healthy volunteers (FIG. 4B).

Neutrophils from healthy and COPD patients were stimulated with LPS (1 μg/ml) in presence of RNO (0.1 nM-1 μM) or DEX (0.1 nM-1 μM) for 6 h and GM-CSF supernatants were measured. The −log IC₅₀ and % of maximum inhibitory effect generated are defined in Table 5. Concentration dependent inhibitory curves are presented in FIG. 4C.

Neutrophil GM-CSF release from COPD patients showed a lower DEX maximal % inhibition than neutrophils from healthy volunteers in response to LPS (Table 5 and FIG. 4C).

TABLE 5
Inhibition of GM-CSF release in isolated peripheral blood neutrophils from
healthy and COPD patients. Inhibitory concentration-dependent curves were
generated by incubation with Roflumilast N-oxide (RNO; 0.1 nM-1 μM) or
Dexamethasone (DEX; 0.1 nM-1 μM) in response to LPS (1 μg/ml). Values
are mean ± SEM of 3 independent experiments run in triplicate. IC₅₀ values
for half-maximum inhibition were calculated by nonlinear regression analysis.

| | HEALTHY | | | COPD | | |
|---|---|---|---|---|---|---|
| Stimulus | Maximal | | | Maximal | | |
| LPS | % Inhibition | −log IC₅₀ | N | % Inhibition | −log IC₅₀ | N |
| RNO | 78.87 ± 6.57 | 8.25 ± 0.21 | 3 | 86.6 ± 8.04 | 8.10 ± 0.22 | 3 |
| DEX | 93.34 ± 5.57 | 7.61 ± 0.11 | 3 | 78.88 ± 5.15* | 7.52 ± 0.13 | 3 |

*p < 0.05 vs. healthy values

In other experiments performed in neutrophils from COPD patients, the combination of RNO (1 nM) and DEX (10 nM) showed an additive effect on the inhibition of GM-CSF release in response to LPS (FIG. 4D).

### EXAMPLE 5

### Results (CCL-5 Release)

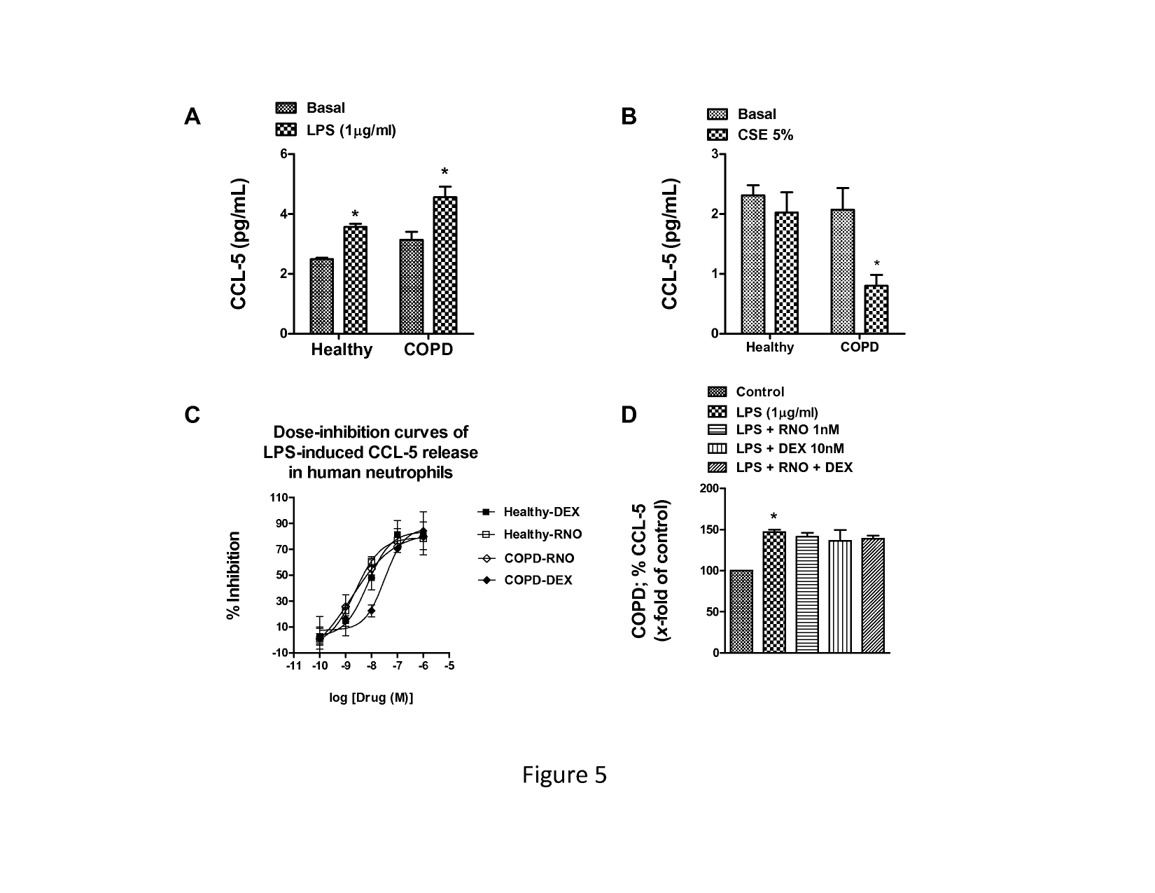

Basal CCL-5 release was not significantly different when measured in neutrophils isolated from COPD patients versus those isolated from healthy volunteers (FIGS. 5A and 5B). LPS (1 μg/ml) increased CCL-5 release (FIG. 5A) while CSE 5% slightly decreased CCL-5 release in both COPD patients and healthy volunteers (FIG. 5B).

Neutrophils from healthy and COPD patients were stimulated with LPS (1 μg/ml) in presence of RNO (0.1 nM-1 μM) or DEX (0.1 nM-1 μM) for 6 h and CCL-5 supernatants were measured. The −log IC₅₀ and % of maximum inhibitory effect generated are defined in Table 6. Concentration dependent inhibitory curves are showed in FIG. 5C.

Neutrophil CCL-5 release from COPD patients was resistant to DEX which reached a −log IC₅₀ of 7.49 M vs 8.14 M in healthy volunteers (Table 6 and FIG. 5C; p<0.05).

TABLE 6
Inhibition of GM-CSF release in isolated peripheral blood neutrophils from
healthy and COPD patients. Inhibitory concentration-dependent curves were
generated by incubation with Roflumilast N-oxide (RNO; 0.1 nM-1 μM) or
Dexamethasone (DEX; 0.1 nM-1 μM) in response to LPS (1 μg/ml). Values
are mean ± SEM of 3 independent experiments run in triplicate. IC₅₀ values
for half-maximum inhibition were calculated by nonlinear regression analysis.

| | HEALTHY | | | COPD | | |
|---|---|---|---|---|---|---|
| Stimulus | Maximal | | | Maximal | | |
| LPS | % Inhibition | −log IC₅₀ | N | % Inhibition | −log IC₅₀ | N |
| RNO | 79.34 ± 3.63 | 8.62 ± 0.14 | 3 | 83.01 ± 6.74 | 8.77 ± 0.35 | 3 |
| DEX | 83.78 ± 8.64 | 8.14 ± 0.25 | 3 | 86.30 ± 10.16 | 7.49 ± 0.24* | 3 |

*p < 0.05 vs. healthy values.

In other experiments performed in neutrophils from COPD patients, the combination of RNO (1 nM) and DEX (10 nM) did not show additive effects (FIG. 5D).

### EXAMPLE 6

### Basal mRNA Expression of Glucocorticoid Resistance Markers in Peripheral Blood Neutrophils from Healthy and COPD Patients

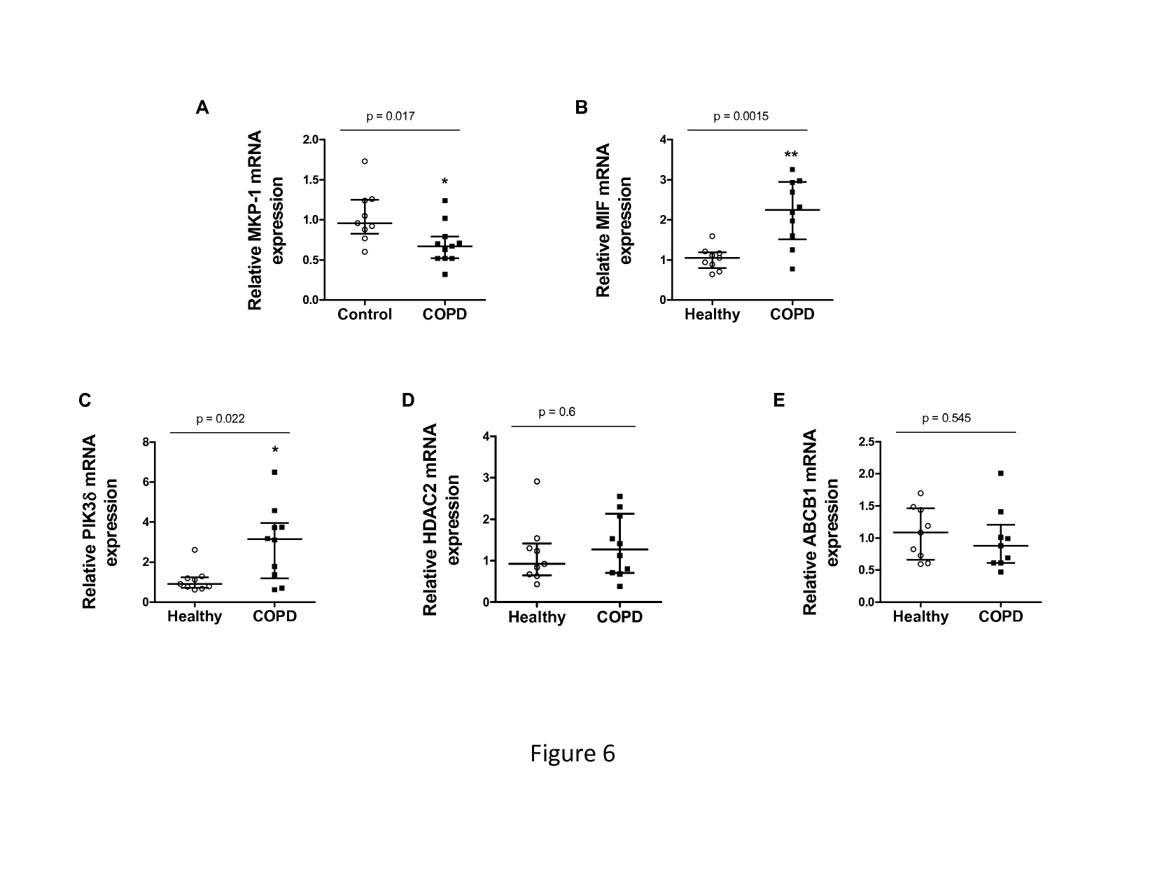

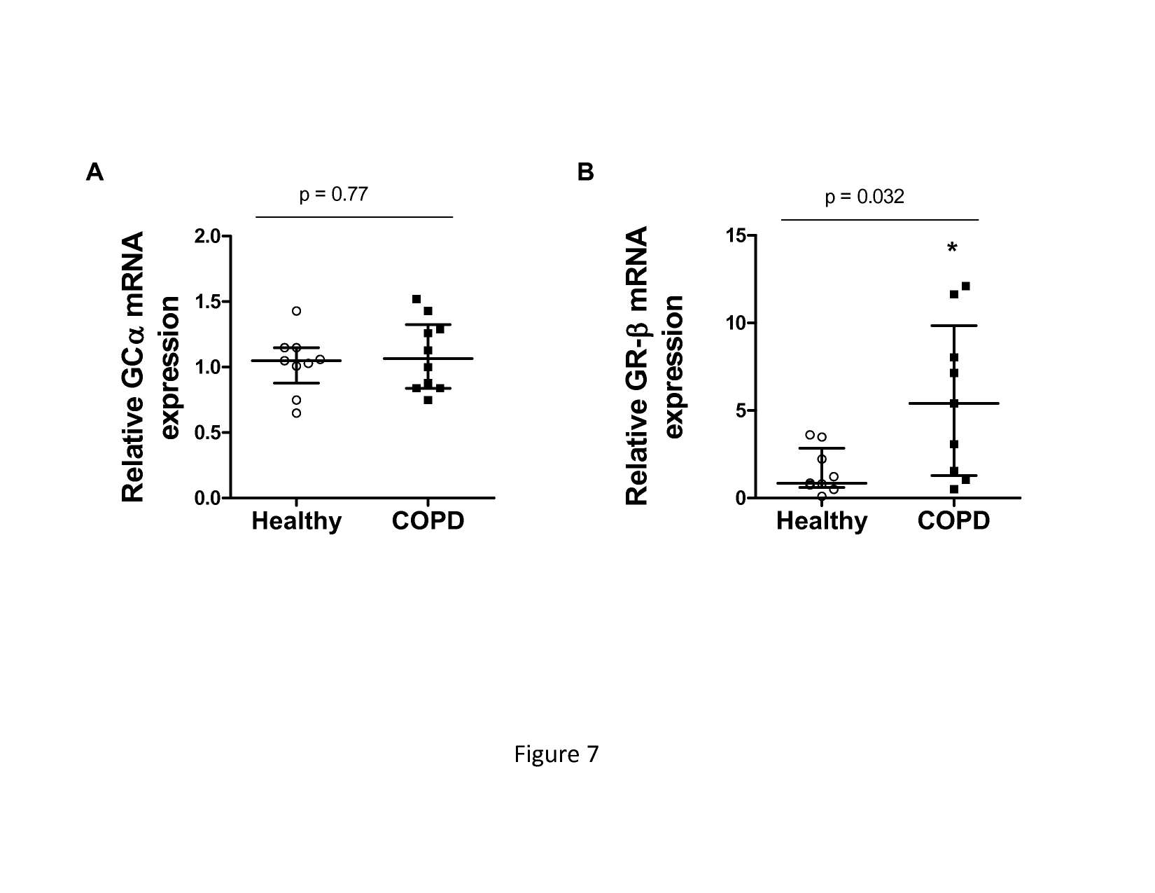

MKP-1 mRNA expression was significantly decreased in neutrophils from COPD patients vs. neutrophils from healthy volunteers (FIG. 6A), while the mRNA expression of MIF was significantly up-regulated in neutrophils from COPD patients (FIG. 6B). mRNA expression of PI3Kδ was significantly increased in neutrophils from COPD patients (FIG. 6C), however the expression of HDAC2 and ABCB1 was similar to that of healthy volunteers (FIGS. 6D and 6E). No significant differences in GCα expression were seen between healthy and COPD patients (FIG. 7A). However the mRNA expression of GCβ was significantly higher in neutrophils from COPD patients (FIG. 7B).

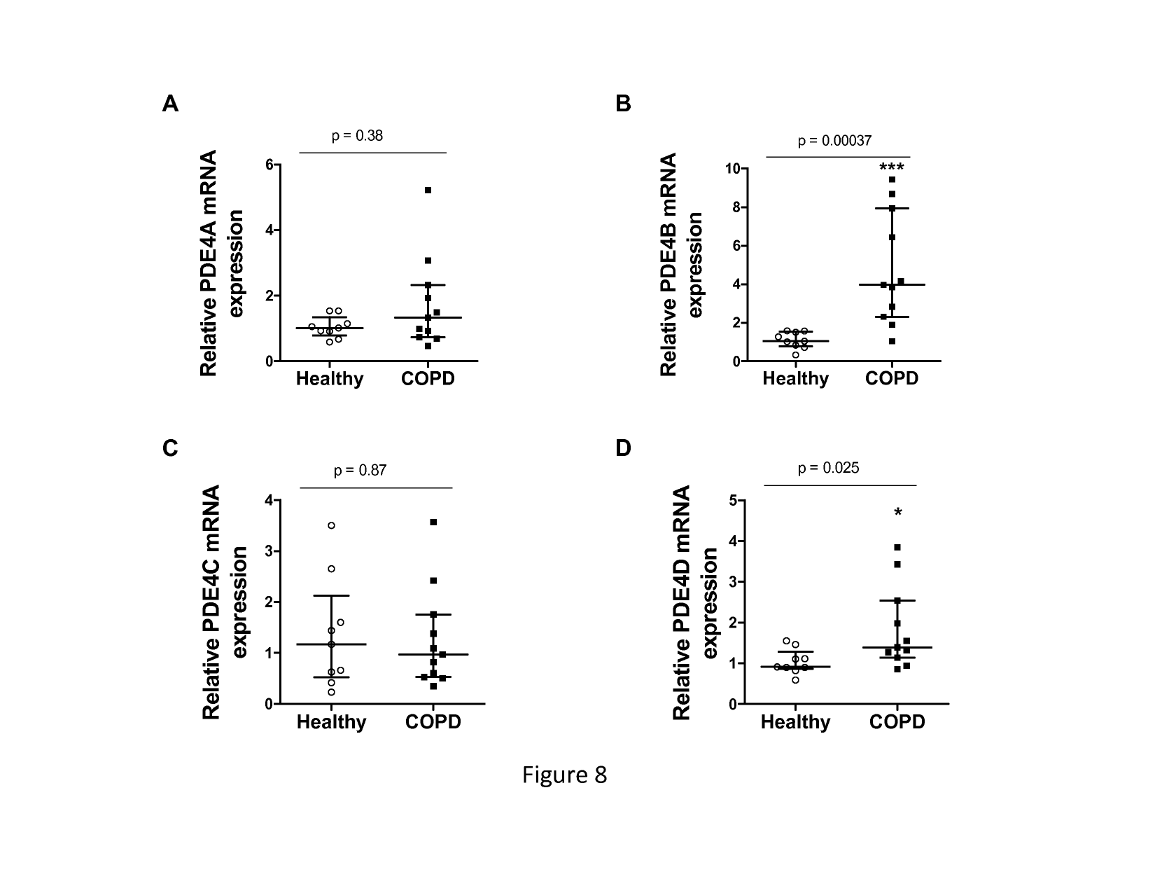

Levels of PDE4 isoforms in neutrophils were also determined since roflumilast N-oxide directly inhibits their activity. From the data obtained here, only PDE4B and D were found to be upregulated in neutrophils from COPD patients (FIGS. 8A-D).

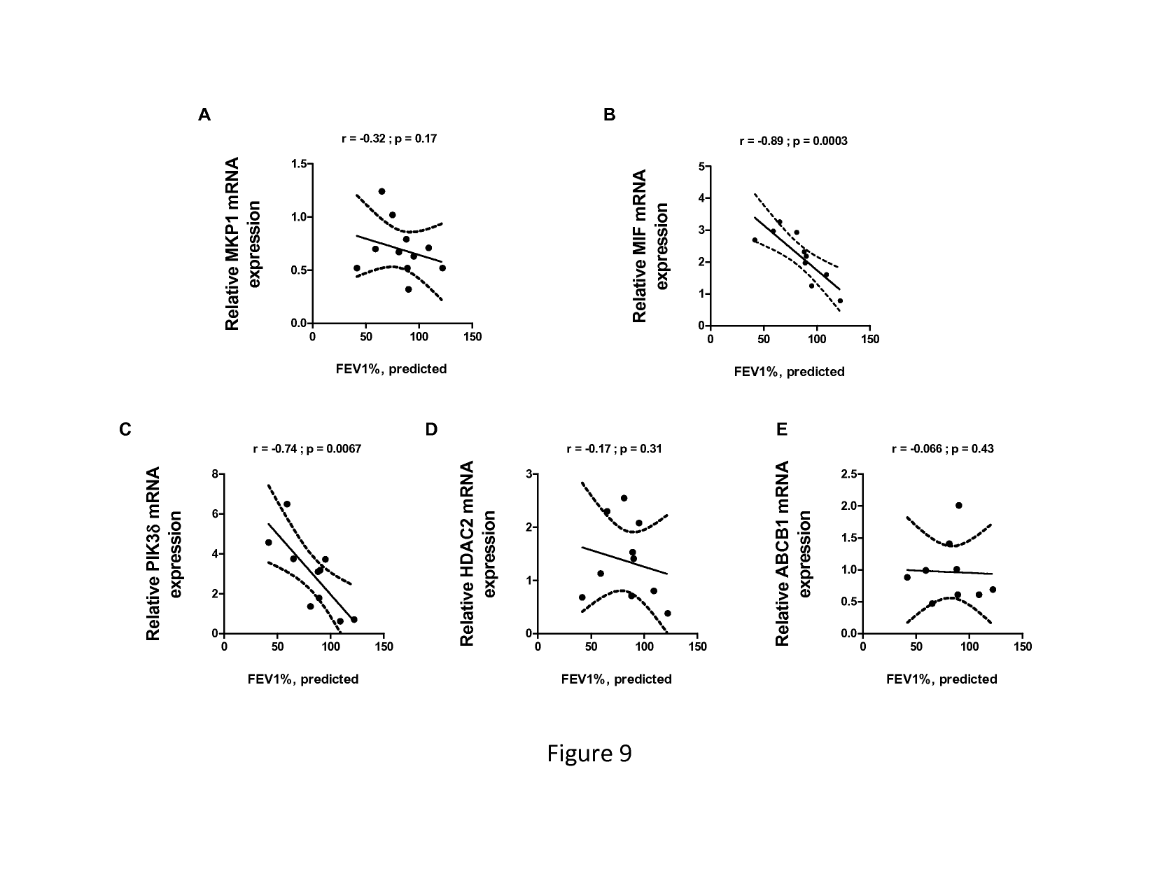

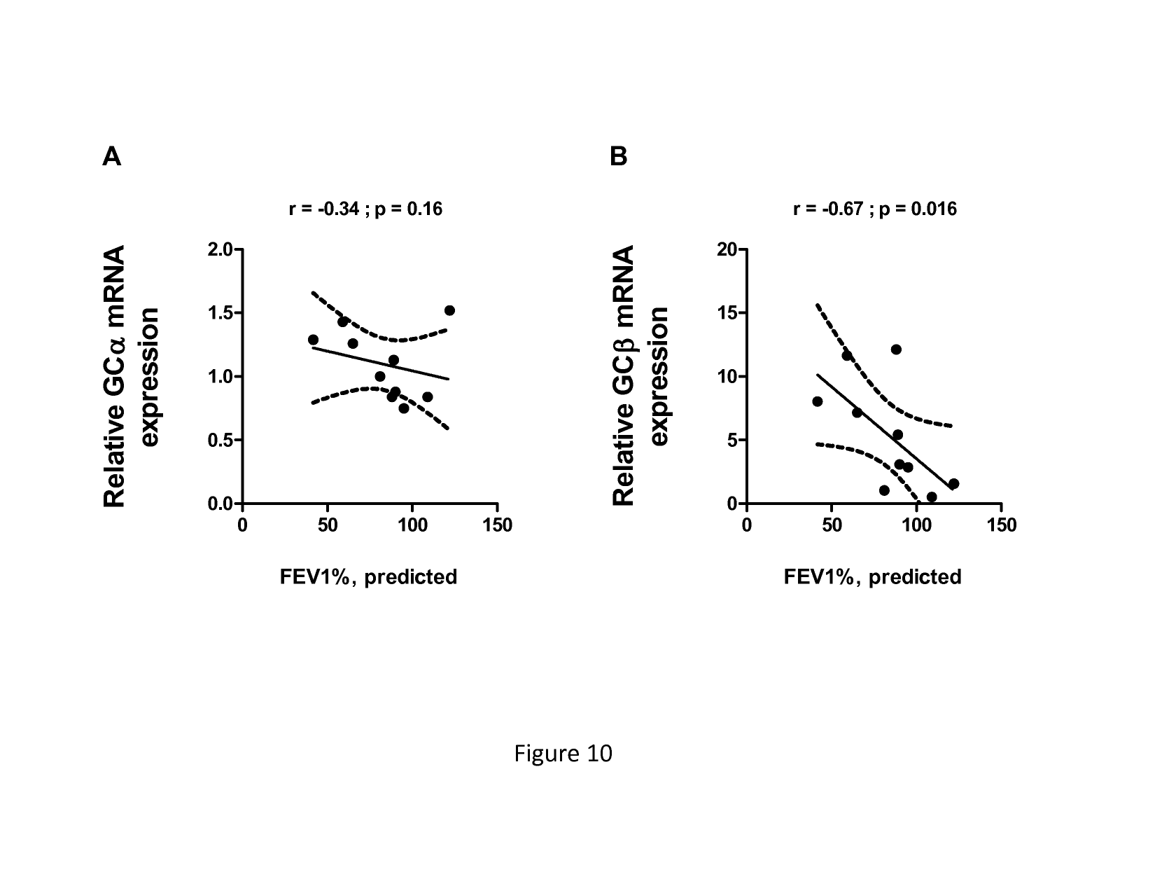

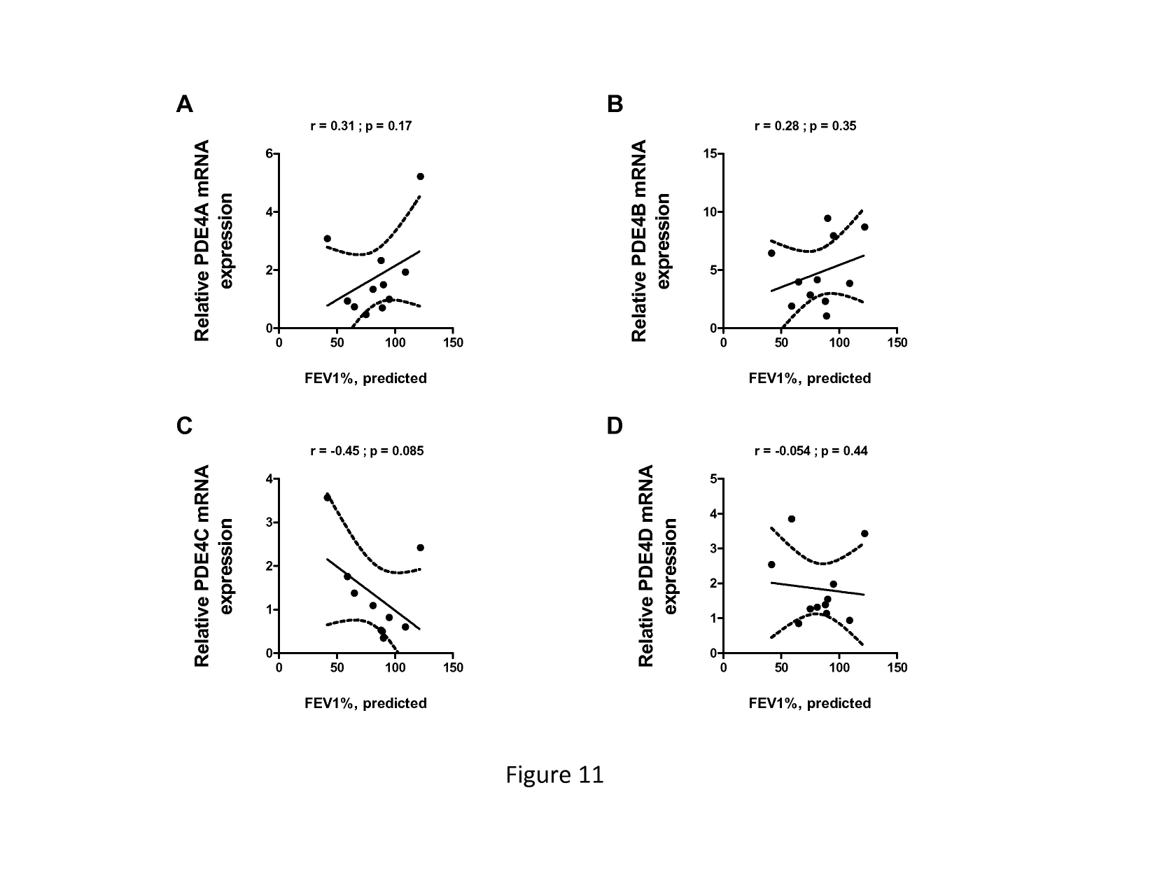

MIF and PI3Kδ neutrophil mRNA expression in COPD patients were inversely correlated with FEV1%, predicted (Spearman r=−0.89 and −0.74; p=0.0003 and 0.0067 respectively) while MKP1, HDAC2 and ABCB1 expression were not correlated with FEV1%, predicted (FIGS. 9A-E). GCβ mRNA expression in neutrophils from COPD patients (but not GCα) was inversely correlated with FEV1%, predicted (Spearman r=−0.67; p=0.016; FIGS. 10A and 10B). PDE4 isoforms expression was not correlated with lung function in COPD patients (FIGS. 11A-D).

### EXAMPLE 7

### Effects of Roflumilast in Reversing Glucocorticoid Resistance in Peripheral Blood Neutrophils from COPD Patients

Neutrophils from COPD patients were exposed to RNO (1 nM and 1 μM), DEX (1 μM), the combination of RNO (1 nM) plus DEX (1 μM), or the antioxidant NAC (1 mM) for 1 h, and stimulated with CSE 5% for 6 h. Then, mRNA was isolated and quantified for different genes.

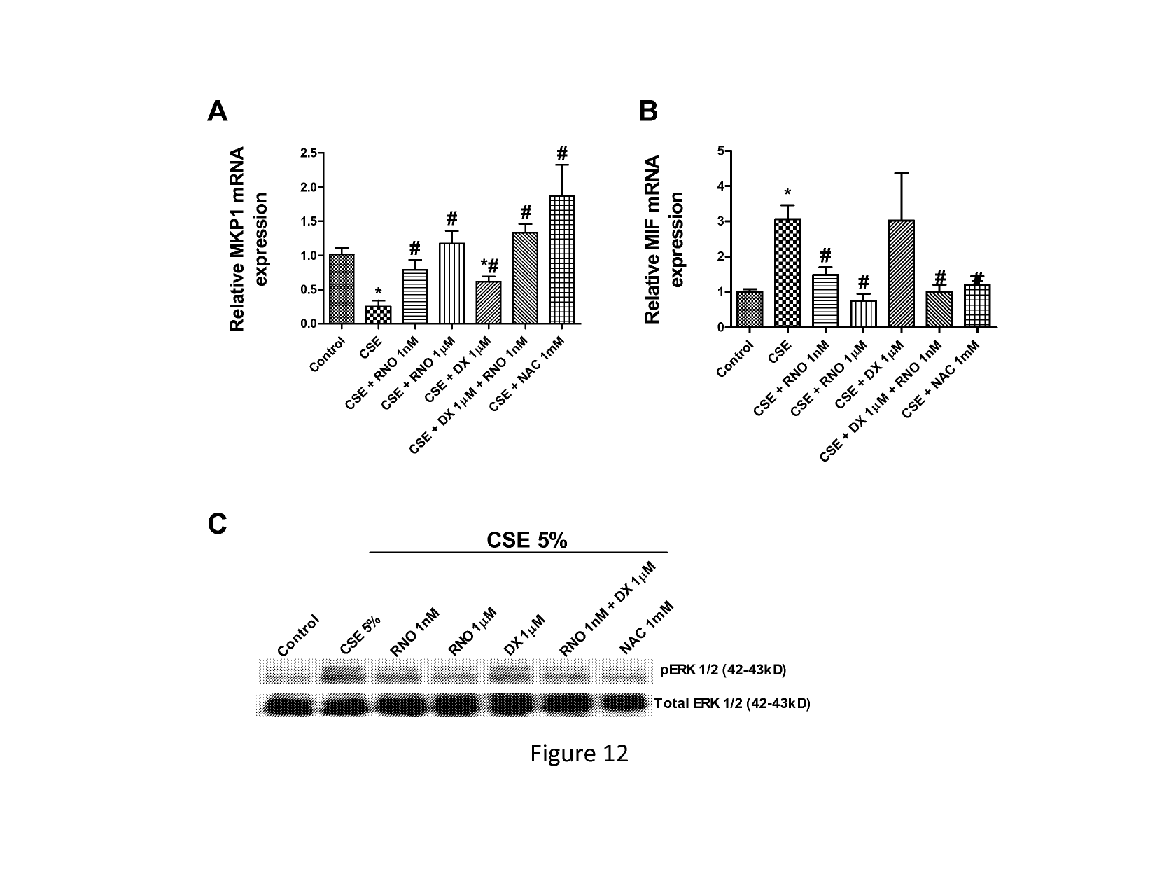

CSE 5% exposure down-regulated MKP1 and increase the MIF mRNA expression. RNO 1 nM and 1 μM reversed the effect of CSE on MKP1 and MIF expression (FIGS. 12A and 12B). DEX 1 μM did not rescue MKP1 to control expression following CSE exposure as observed for MIF expression (FIGS. 12A and 12B). The combination of RNO and DEX showed an additive effect reversing the effect of CSE on MKP1 and MIF expression. Furthermore, the antioxidant NAC effectively increased MKP1 and reduced the MIF mRNA expression, suggesting a potential role of ROS in this process.

Since DEX may activate MKP 1, and MKP 1 inactivates p-ERK1/2 and therefore cytokine release, cells displaying corticoid resistance (such as neutrophils from COPD patients) may potentially show a lack of effect of DEX on phosphorylation of ERK1/2.

In this regard, neutrophils from COPD patients were incubated with RNO (1 nM and 1 μM), DEX (1 μM), the combination of RNO (1 nM) plus DEX (1 μM), or the antioxidant NAC (1 mM) for 1 h, and stimulated with CSE 5% for 20 min. CSE 5% increased the phosphorylation of ERK1/2 that was effectively inhibited by RNO (1 nM and 1 μM), but not by DEX (1 μM). In contrast, the combination of RNO (1 nM) plus DEX (1 μM) reversed the DEX resistance on ERK1/2 phosphorylation (FIGS. 12A and 12B). The antioxidant NAC also inhibited the CSE-induced ERK1/2 phosphorylation (FIG. 12C).

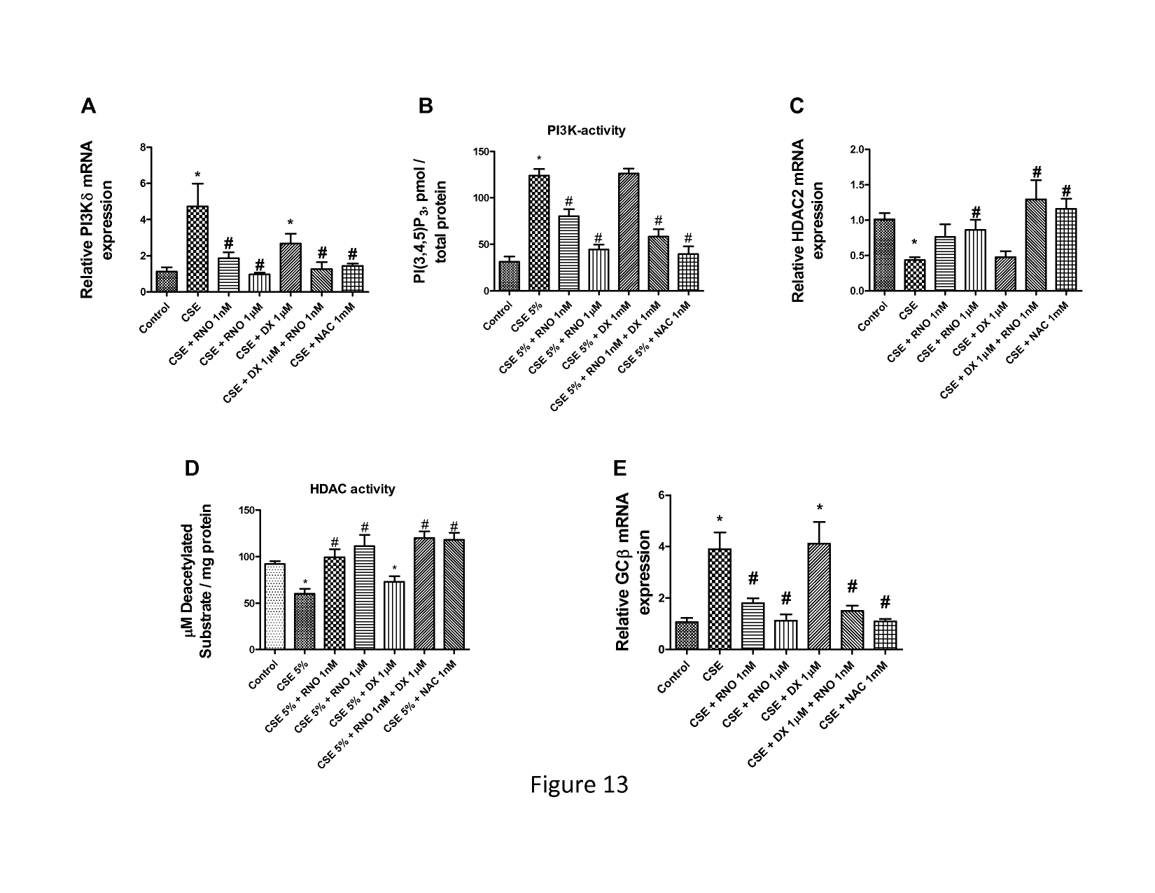

In other experiments, CSE 5% incubation, increased the PI3Kδ expression and PI3K activity, increased GCβ mRNA expression, and decreased the HDAC2 expression and HDAC activity in neutrophils from COPD patients. RNO (1 nM and 1 μM) but not DEX (1 μM) inhibited the CSE induced PI3Kδ and GCβ upregulation as well as the CSE-induced HDAC2 downregulation (FIG. 13A-E). In contrast, the combination of RNO (1 nM) plus DEX (1 μM) reversed the DEX resistance on PI3Kδ and GCβ upregulation (FIGS. 13A and 13D) and HDAC2 downregulation (FIGS. 13B and 13C).

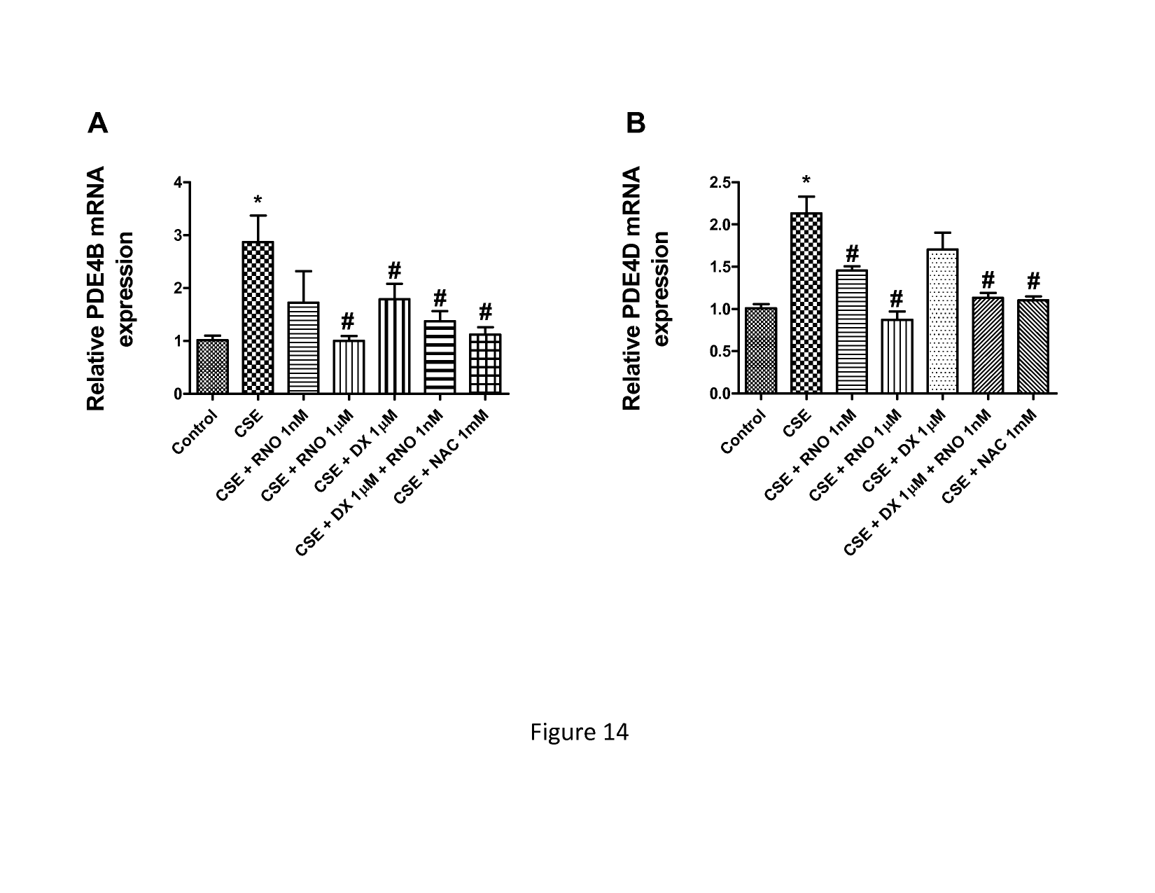

The antioxidant NAC also inhibited the CSE-induced PI3Kδ and GCβ mRNA up-regulation and HDAC2 downregulation. Similar results were found for CSE-induced PDE4B and PDE4D mRNA expression (FIGS. 14A and 14B).

Based on these results and a number of previous reports, there appears to be a relationship between reactive oxygen species (ROS) or oxidative stress burden with glucocorticoid resistance and with the activation of the different intracellular pathways related with glucocorticoid resistance. Roflumilast shows potent antioxidant properties on human neutrophils. Therefore, the inhibition of DEX resistance that is observed in neutrophils from COPD patients could be due to its antioxidant properties. Furthermore, ROS may also activate PI3Kδ/Akt pathway, downregulate HDAC2, increase MIF and downregulate MKP1. Thus, it was imperative to explore the role of RNO on intracellular ROS of COPD neutrophils.

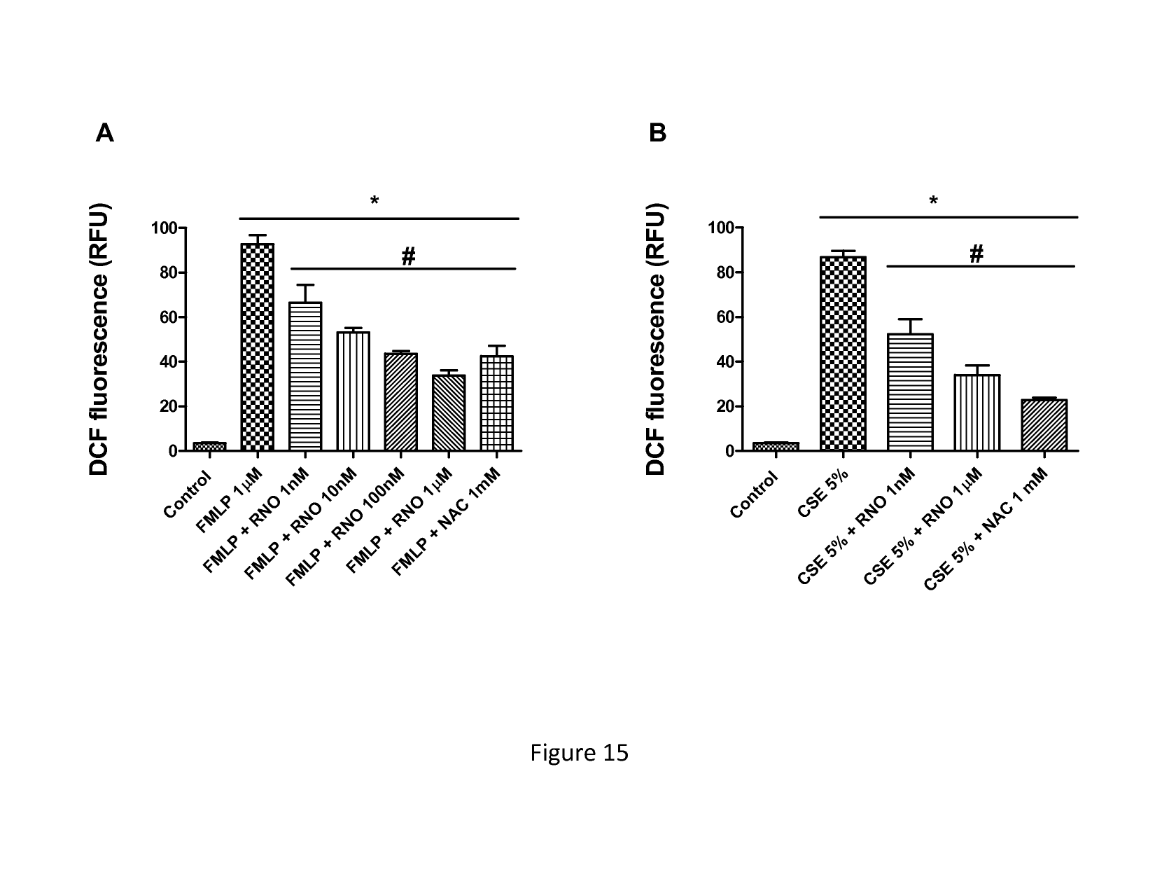

To this end, whole blood was stimulated with the bacterial peptide fMLP 1 μM or with CSE 5% in presence or absence of RNO or NAC for 1 h. RNO dose-dependently inhibited ROS generation in selected neutrophils from COPD patients as occurs with NAC (FIGS. 15A and 15B).

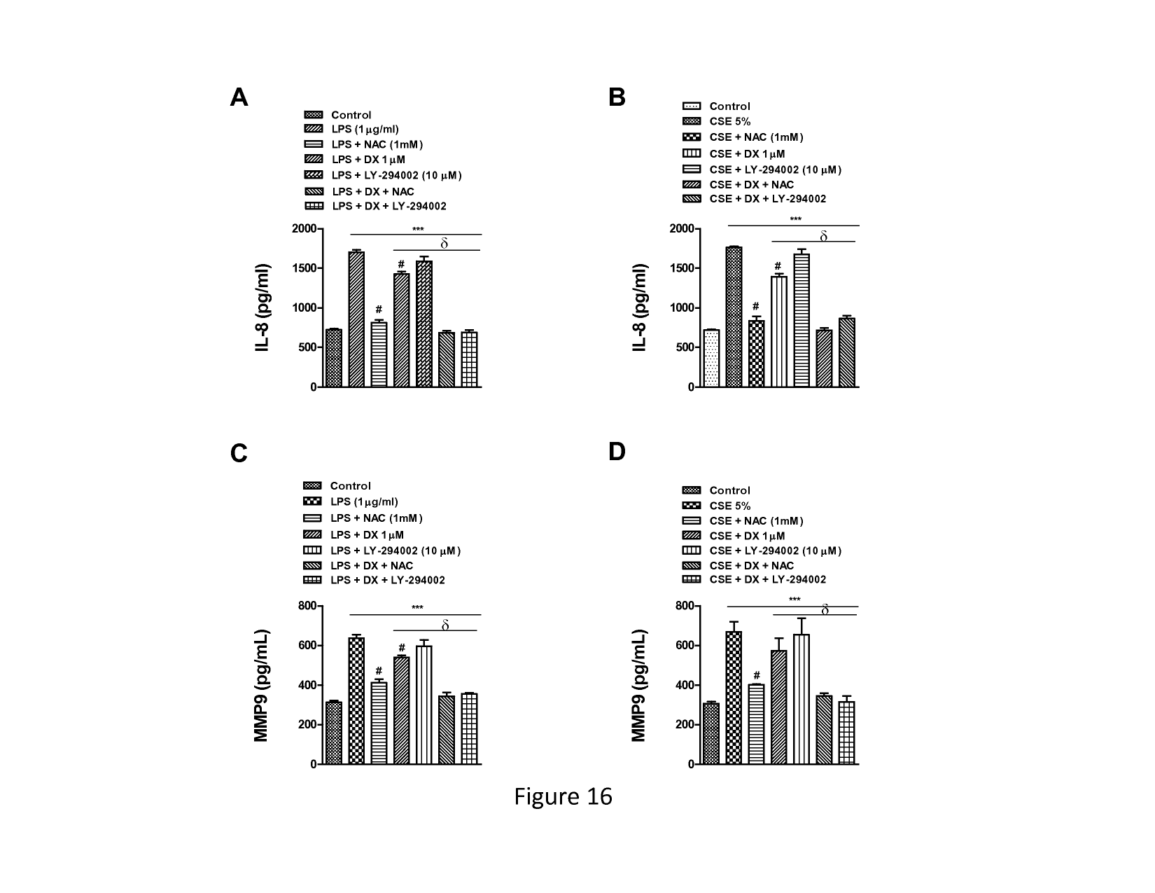

Since PI3Kδ and ROS are both related with the generation of resistance to glucocorticoids, the pan-inhibitor of PI3K, LY-294002 and the antioxidant NAC were studied for their ability to reduce the glucocorticoid resistant to IL-8 and MMP-9 release in neutrophils from COPD patients stimulated with LPS or CSE. Both LY-294002 and NAC were able to rescue DEX anti-inflammatory effect on IL-8 and MMP-9 release, confirming this hypothesis (FIG. 16A-D).

In summary, peripheral neutrophils from COPD patients are resistant to the anti-inflammatory properties of the glucocorticoid dexamethasone with respect to the LPS or CSE-induced IL-8 and MMP-9 release, and to a lesser extent, respect to the release of IL-1β, GM-CSF and CCL-5. Roflumilast N-oxide restores the anti-inflammatory properties of DEX in neutrophils from COPD patients for IL-8, MMP-9, IL-113 and GM-CSF. Neutrophils from COPD patients showed an increased expression of glucocorticoid resistant markers MIF, PI3Kδ, GCβ, and down-regulation of HDAC-2 and MKP 1. In addition, PDE4 isoforms PDE4B and PDE4D were found to be upregulated in neutrophils from COPD patients.

The COPD neutrophil expression of MIF, PI3Kδ and GCβ were inversely correlated with the FIV1, % predicted which is in agreement with the increased glucocorticoid resistance in parallel to COPD severity.

Roflumilast N-oxide inhibits the intracellular ROS induced by CSE in neutrophils from COPD patients, which may explain why RNO restore the control levels of glucocorticoid markers MIF, PI3Kδ, GCβ, HDAC-2 and MKP1 and allows Dexamethasone to exert its anti-inflammatory properties.

Thus, in addition to the clinical advantage of roflumilast N-oxide in COPD, the combination of roflumilast N-oxide with glucocorticoids could contribute to rescue the anti-inflammatory properties of glucocorticoids impaired in COPD patients.

### EXAMPLE 8

The efficacy of PDE4 inhibitors combined with corticosteroids will be examined on different functional and mechanistic outputs, e.g., secretion and expression of inflammatory mediators resistant to corticosteroids, or enzymes, or transcription factors in an in-vivo model, e.g., murine, relevant to the pathogenic mechanisms of COPD.

All studies will involve 6 mice per treatment group. Mice will be exposed continuously to whole body cigarette smoke for 5 days. Throughout this period, groups will receive roflumilast, dexamethasone, or both at doses selected for determination of therapeutic effect. In this study the roflumilast doses will be 0.05, 0.1, 0.3, 1 and 5 mg/kg per day delivered by oral gavage and the dexamethasone doses will be 0.05, 0.1, 0.3, 1 and 5 mg/kg per day delivered by intraperitoneal injection. In all cases, at the end of 5-day exposure pulmonary function tests (functional residual capacity, total lung capacity, and compliance) will be performed. Mice will be euthanized and blood collected by cardiac puncture for determination of roflumilast and roflumilast N-oxide levels. BAL fluid will then be collected and processed for determination of total and differential cell count by flow cytometry. Infiltration of inflammatory cells into lung tissue, and consequently into BAL fluid will be analyzed for the presence of inflammatory markers.

Alveolar macrophages will be isolated from BAL fluid of mice continuously exposed to cigarette smoke for 5 days and will be similarly isolated from BAL fluid of COPD patients. In one set of experiments, mouse cells will be treated for 24 or 48 h with or without roflumilast and with or without cigarette smoke extract (CSE; 1% v/v). In a second set of experiments, cells from mice and COPD patients will be treated for the identified time period with roflumilast, dexamethasone, or a combination of the two ingredients.

### Preparation of CSE

CSE was prepared as previously described.

### Murine Alveolar Macrophage (AM) Isolation

After euthanasia a tracheal cannula will be inserted and 1 ml followed by 4 ml of phosphate-buffered saline (PBS) containing 0.05 mM EDTA will be instilled. The lavage fluid will then be recovered by gentle aspiration. The lavage fractions will be pooled and centrifuged at 300×g for 10 min at 4° C. The cell pellet will then be washed twice. Total cell number will be counted using a hemocytometer. Differential cell counts will be performed on cytospin and stained with Diff-Quick stain (Siemens, Newark, Del.). At least 200 cells will be counted and identified according to morphological criteria. Cells will then resuspended at 10⁶ cell/ml in RPMI plus 10% FBS and seeded onto tissue culture-treated plates or dishes. After 2 h, plates/dishes are then washed three times with PBS and the supernatant containing non-adherent cells is discarded. Adherent cells are resuspended in fresh culture medium. Freshly isolated cells will be used in some studies (obtained only from Atlanta VAMC), but RNA and protein from AMs will also be made.

### Human AM Isolation

BAL fluid will be obtained from COPD and normal subjects using standard bronchoscopic methods on the 2^{nd} floor of the Atlanta VAMC, delivered on ice by staff to our lab on the 12^{th} floor, and processed immediately. Briefly, 180 ml of sterile saline fluid is instilled in the right middle lobe in 60 ml aliquots, aspirated, and then filtered through sterile gauze. On occasion, the lingula of the left lung will be lavaged as the sole site or additionally with the right middle lobe. Cells are pelleted by centrifuging at 300×g for 10 min at 4° C. and washed twice with PBS. Total cell number will be counted using a hemocytometer. Differential cell counts will be performed on cytospin and stained with Diff-Quick stain (Siemens, Newark, Del.). At least 200 cells will be counted and identified according to morphological criteria. Cells will then resuspended at 10⁶ cell/ml in RPMI plus 10% FBS and plated onto tissue culture-treated plates or dishes. After 2 h, plates/dishes are then washed three times with PBS and the supernatant containing non-adherent cells is discarded. Adherent cells are resuspended in fresh culture medium. Freshly isolated cells will be used in some studies (obtained only from Atlanta VAMC), but RNA and protein from AMs will also be made. On average, 2-3 samples are obtained per week.

### Flow Cytometry

Cells from whole lung or BAL fluid will be analyzed by flow cytometry by a modification of previously reported methods. Briefly, cells will be treated with an F_{c} receptor blocking agent (anti-CD16/CD32) to reduce nonspecific binding, then with monoclonal antibodies specific to each cell of interest. Cells will be counted with a Becton-Dickson FACS Calibur flow cytometer (BD Biosciences, San Jose, Calif.). Macrophages will be distinguished from dendritic cells (also CD11c-bright) by their high autofluorescence. Cell suspensions will be incubated with 7-amino-actinomycin to exclude dead cells. A minimum of 10,000 viable cells will be analyzed.

### Pulmonary Function Tests

Briefly, mice will be anesthetized and a 19-guage tracheostomy tube inserted. Mice will then be maintained on a mechanical ventilator in a sealed plethysmograph, part of the Pulmonary Maneuvers System (Buxco Electronics, Wilmington, N.C.). Functional reserve capacity (FRC) will be measured by occluding the airway while the anesthetized mouse attempts to breathe spontaneously, with Boyle's law used to calculate FRC from data on exerted pressure and changes in thoracic volume. Compliance and total lung capacity will be measured using a quasistatic pressure/volume maneuver.

### EXAMPLE 9

The efficacy of PDE4 inhibitors combined with montelukast will be examined in an in-vivo model, e.g., murine, primates, etc., relevant to leukotriene-mediated bronchoconstriction.

Rats will be sensitized to bronchoconstriction by injecting a dose of allergen for 12-24 days. Prior to challenge, the treatment groups will receive roflumilast, montelukast, or both at doses selected for determination of a therapeutic effect. The animals will be challenged with aerosol doses of LTD₄. Following challenge, data will be calculated as a percent change from control values for each respiratory parameter including airway resistance. The results for the treatment groups will be subsequently obtained for a period of 60 minutes post challenge and compared to baseline control values to determine percent inhibition of symptoms.

Having thus described in detail advantageous embodiments of the present invention, it is to be understood that the invention defined by the above paragraphs is not to be limited to particular details set forth in the above description as many apparent variations thereof are possible without departing from the spirit or scope of the present invention. Modifications and variations of the methods described herein will be obvious to those skilled in the art and are intended to be encompassed by the following claims.

What is claimed is:

1. A pharmaceutical composition comprising roflumilast or a pharmaceutically acceptable salt, ester, prodrug, or pharmaceutically acceptable salts of esters or prodrugs, or N-oxide thereof in combination with a corticosteroid or a pharmaceutically acceptable salt, ester, prodrug, or pharmaceutically acceptable salt of ester or prodrug thereof and a pharmaceutically acceptable excipient.

2. The composition of claim 1, wherein the corticosteroid is selected from the group consisting of dexamethasone, prednisone and budesonide.

3. The composition of claim 2, wherein the corticosteroid is budesonide.

4. A pharmaceutical composition comprising roflumilast or a pharmaceutically acceptable salt, ester, prodrug, or pharmaceutically acceptable salts of esters or prodrugs, or N-oxide thereof in combination with a leukotriene receptor antagonist or a pharmaceutically acceptable salt, ester, prodrug, or pharmaceutically acceptable salt of ester or prodrug thereof and a pharmaceutically acceptable excipient.

5. The composition of claim 4, wherein the leukotriene receptor antagonist is montelukast.

6. The composition of claim 4, comprising 500 μg of the roflumilast or pharmaceutically acceptable salt, ester, prodrug, or pharmaceutically acceptable salts of esters or prodrugs, or N-oxide thereof.

7. The composition of claim 4, comprising 10 mg of the leukotriene receptor antagonist or pharmaceutically acceptable salt, ester, prodrug, or pharmaceutically acceptable salt of ester or prodrug thereof.

8. An oral dosage form comprising the composition of claim 2.

9. An oral dosage form comprising the composition of claim 4.

10. A method of treating COPD by administering to a subject an oral dosage form comprising roflumilast or a pharmaceutically acceptable salt, ester, prodrug, or pharmaceutically acceptable salts of esters or prodrugs, or N-oxide thereof in combination with a corticosteroid or a pharmaceutically acceptable salt, ester, prodrug or pharmaceutically acceptable salt of ester or prodrug thereof.

11. The method of claim 8, wherein the oral dosage form comprises 500 μg of roflumilast or a pharmaceutically acceptable salt, ester, prodrug, or pharmaceutically acceptable salts of esters or prodrugs, or N-oxide thereof.

12. The method of claim 8, wherein the corticosteroid is selected from the group consisting of dexamethasone, prednisone and budesonide.

13. The method of claim 10, wherein the corticosteroid is budesonide.

14. A method of restoring steroid sensitivity in a subject suffering from COPD by administering to a subject an oral dosage form comprising roflumilast or a pharmaceutically acceptable salt, ester, prodrug, or pharmaceutically acceptable salts of esters or prodrugs, or N-oxide thereof in combination with a corticosteroid or a pharmaceutically acceptable salt, ester, prodrug, or pharmaceutically acceptable salt of ester or prodrug thereof.

15. The method of claim 12, wherein the oral dosage form comprises 500 μg of roflumilast or a pharmaceutically acceptable salt, ester, prodrug, or pharmaceutically acceptable salt of ester or prodrug, or N-oxide thereof.

16. The method of claim 12, wherein the corticosteroid is selected from the group consisting of dexamethasone, prednisone and budesonide.

17. The method of claim 14, wherein the corticosteroid is budesonide.

18. A method of treating COPD by administering to a subject an oral dosage form comprising roflumilast or a pharmaceutically acceptable salt, ester, prodrug, or pharmaceutically acceptable salt of ester or prodrug, or N-oxide thereof in combination with a leukotriene receptor antagonist or a pharmaceutically acceptable salt, ester, prodrug or pharmaceutically acceptable salt of ester or prodrug thereof.

19. The method of claim 18, wherein the oral dosage form comprises 500 μg of roflumilast or a pharmaceutically acceptable salt, ester, prodrug, or pharmaceutically acceptable salt of ester or prodrug, or N-oxide thereof.

20. The method of claim 18, wherein the leukotriene receptor antagonist is montelukast or pharmaceutically acceptable salt, ester, prodrug or pharmaceutically acceptable salt of ester or prodrug thereof.

21. The method of claim 20, wherein the oral dosage form comprises 10 mg of montelukast or pharmaceutically acceptable salt, ester, prodrug or pharmaceutically acceptable salt of ester or prodrug thereof.
